(19) United States
(12) Patent Application Publication
(10) Pub. No.: US 2025/0352647 A1
(43) Pub. Date: Nov. 20, 2025

(54) METHOD FOR ENHANCING DURABILITY OF IMMUNE CELL

(71) Applicant: TSINGHUA UNIVERSITY, Beijing (CN)

(72) Inventors: Min PENG, Beijing (CN); Lixia WANG, Beijing (CN); Gang JIN, Beijing (CN); Guangyue ZHANG, Beijing (CN)

(21) Appl. No.: 18/842,679

(22) PCT Filed: Jun. 17, 2022
(86) PCT No.: PCT/CN2022/099498

§ 371 Date: Jul. 14, 2025

(30) Foreign Application Priority Data
Nov. 10, 2021 (CN) 202111324605.2

(51) Int. Cl. A61K 40/11; A01K 67/00; A61K 35/15; A61K 35/17; A61K 40/13; A61K 40/15; A61K 40/31; A61K 40/32; C12N 5/0781; C12N 5/0783; C12N 5/0787; C12N 5/10; C12N 9/22; C12N 15/113

(52) U.S. Cl. CPC A61K 40/11; A01K 67/00; A61K 35/15; A61K 35/17; A61K 40/13; A61K 40/15; A61K 40/31; A61K 40/32; C12N 5/0635; C12N 5/0636; C12N 5/0642; C12N 5/0646; C12N 5/10; C12N 9/222; C12N 15/1135; A01K 2267/03; C12N 2310/20; C12N 2510/00

(57) ABSTRACT

The present invention provides a recombinant immune cell and the preparation method, the gene regulation system and the use thereof. By reducing or eliminating the expression and/or biological functions thereof of the BCOR gene and the ZC3H12A gene, the persistence of the recombinant immune cell is enhanced. In some embodiments, the present invention obtains CAR-T cells with knockout of double genes ZC3H12A and BCOR by gene editing, which can persist in vivo, solving the technical problem of long-term effectiveness of CAR-T treatment. In some embodiments, the gene-edited CAR-T cells persist in vivo and can continuously secrete therapeutic biological molecules, achieving the purpose of long-term effectiveness of a single administration.

### SEQUENCE LISTING

The instant applicant contains a Sequence Listing which has been submitted electronically in ASCII format and is hereby incorporated by reference in its entirety. Said ASCII copy, created on Jun. 12, 2025, is named 51728-016001_Sequence_Listing_6_12_25_ST25 and is 31,974 bytes in size.

### TECHNICAL FIELD

The present invention belongs to the field of cell technology, and relates to a recombinant immune cell with simultaneous knockout of ZC3H12A and BCOR genes and the preparation method, the gene regulation system and the use thereof, specifically relates to a T cell with enhanced T cell persistence and stable implantation in vivo and having secretory function, and the preparation method, the gene regulation system and the use thereof.

### BACKGROUND TECHNOLOGY

Adoptive cell transfer therapy includes chimeric antigen receptor (CAR)-T and T cell receptor (TCR)-T, etc., which have very significant effects in tumor immunotherapy, especially for lymphocytic leukemia.

At present, CAR-T therapy has two defects: on the one hand, patients need to be pretreated with chemotherapy before CAR-T cells are infused back, otherwise the infused CAR-T cells cannot effectively expand, additionally the chemotherapy has great toxic or side effects; on the other hand, the back-infused CAR-T cells have a limited duration in vivo, and many patients will therefore relapse. In the adoptive cell transfer therapy, how to improve the persistence of recombinant immune cells is a technical problem that needs to be solved urgently.

The human BCOR gene ((Gene ID: 54880, updated on May 29, 2022, https://www.ncbi.nlm.nih.gov/gene/54880) and the mouse Bcor gene (Gene ID: 71458, updated on May 22, 2022, https://www.ncbi.nlm.nih.gov/gene/71458) encode the transcriptional repressor BCOR in cells. The human ZC3H12A gene (Gene ID: 80149, updated on May 22, 2022, https://www.ncbi.nlm.nih.gov/gene/80149) and the mouse Zc3h12a gene (Gene ID: 230738, updated on May 22, 2022, https://www.ncbi.nlm.nih.gov/gene/230738) encode the protein ZC3H12A involved in mRNA degradation in cells. The above genes are all incorporated into the present invention by reference.

CN113151178A discloses a recombinant T cell knocking out the Rc3h1 gene and/or the Zc3h12a gene and the uses thereof. The T cells knocking out Rc3h1 and/or Zc3h12a do not last more than 1 month in vivo, and the therapeutic effect is poor, requiring repeated infusion of recombinant T cells. WO2020163365A2 discloses a recombinant T cell that reduces the expressions and/or functions of at least two endogenous target genes selected from SOCS1, PTPN2 and ZC3H12A. At present, there is no prior art in which recombinant immune cells that simultaneously target the Bcor gene and the Zc3h12a gene are used for treatment of diseases or as a carrier.

### SUMMARY OF THE INVENTION

The present invention found that reducing or eliminating the BCOR gene or ZC3H12A gene alone cannot confer persistence or immortal-like properties to recombinant immune cells. Based on this discovery, the present invention provides a recombinant immune cell and the preparation method, the gene regulation system and the use thereof. By reducing or eliminating the expressions and/or the biological functions thereof of the BCOR gene and the ZC3H12A gene, the persistence of the recombinant immune cell of the present invention is enhanced, and the recombinant immune cell is endowed with extremely strong stemness or immortal-like and functional properties.

One object of the present invention is to provide a recombinant immune cell, the expressions and/or functions of the BCOR gene and the ZC3H12A gene thereof are reduced or eliminated.

Another object of the present invention is to provide a method for preparing the recombinant immune cell of the present invention, including treating the BCOR gene and the ZC3H12A gene in the recombinant immune cell with a gene knockout technology, a gene silencing technology or an inactivation mutation technology or a small molecule inhibitor.

Another object of the present invention is to provide a gene regulation system for preparation of the recombinant immune cell of the present invention.

Another object of the present invention is to provide a kit comprising the gene regulation system of the present invention.

Another object of the present invention is to provide a method for producing the recombinant immune cells of the present invention.

Another object of the present invention is to provide a composition for treating diseases, which comprises the recombinant immune cells or gene regulation system of the present invention.

Another object of the present invention is to provide a method for treating a disease or condition in a subject in need thereof, comprising administering to the subject the recombinant immune cells, composition or gene regulation system of the present invention to treat the subject's immune cells.

Another object of the present invention is to provide a use of the recombinant immune cells, the composition and the gene regulation system of the present invention for treating the subject's immune cells in the manufacture of a medicament for treating a disease or condition.

Another object of the present invention is to provide a use of the recombinant immune cells of the present invention as a carrier for stably delivering biological molecules for treating a disease.

Another object of the present invention is to provide a use of reducing or eliminating the expressions and/or functions of the BCOR gene and the ZC3H12A gene in immune cells. The use includes increasing the stemness of immune cells, inhibiting the exhaustion of immune cells, promoting the expansion of immune cells, conferring memory to immune cells, prolonging the persistence of immune cells, and increasing the self-renewal ability of immune cells.

Another object of the present invention is to provide a method for producing an animal model, which uses the preparation method of the present invention to treat the immune cells of an animal or uses the gene regulation system of the present invention to introduce the immune cells of an animal, or uses the kit of the present invention to treat the immune cells of an animal.

Another object of the present invention is to provide an animal model, which is produced using the method of the present invention.

One object of the present invention is to provide a recombinant T cell.

The recombinant T cells provided by the present invention do not comprise the BCOR gene and the ZC3H12A gene, or the biological functions of the BCOR gene products and the ZC3H12A gene products of the recombinant T cells are inhibited or repressed.

The above-mentioned recombinant T cells are recombinant T cells obtained by knocking out the BCOR gene and the ZC3H12A gene of the target T cells, and introducing a CAR structure or TCR structure or other corresponding structures of the adoptive cell transfer therapy with a target.

In the above-mentioned recombinant T cells, the target T cells are CD8 T cells or other types of T cells.

In the above-mentioned recombinant T cells, the CAR structure with a target is a CD19-CAR structure or a CAR or TCR structure that recognizes other targets.

In the above-mentioned recombinant T cells, the knockout is to knock out the BCOR gene and ZC3H12A gene of the target T cells by CRISPR-Cas9 method or other methods, or to inhibit the functions of BCOR gene products and ZC3H12A gene products by other methods.

In the above-mentioned recombinant T cells, when the BCOR gene in the target T cells is knocked out by CRISPR-Cas9 method, the target sequence targeting the BCOR gene is SEQ ID NO: 3; when the ZC3H12A gene in the target T cells is knocked out by CRISPR-Cas9 method, the target sequence targeting the ZC3H12A gene is SEQ ID NO:4.

Further, the recombinant cells are obtained by introducing the carrier carrying the target sequence targeting the BCOR gene, the target sequence targeting the ZC3H12A gene, and the structure expressing CD19-CAR when knocking out into the target T cells.

In the examples of the present invention, the recombinant cell is a cell obtained by introducing pMSCV-hU6-sgBcor-hU6-sgZc3h12a-EFS-Thy1.1-P2A-CD19-CAR or pMSCV-hU6-sgBcor-mU6-sgZc3h12a-EFS-Thy1.1-P2A-humanized CD19-CAR into a target CD8 T cell, wherein the target CD8 T cell is derived from the CD8 T cells isolated and obtained from the spleen of a Cas9 transgenic mouse (from Jaxson Laboratory, Stock No: 026430).

The above-mentioned recombinant T cells also comprise a gene expressing a corresponding molecule for treating a disease. In the examples of the present invention, the corresponding molecules for treating diseases take the IL23R fusion protein for treating enteritis as an example.

Another object of the present invention is to provide a method for preparing the above-mentioned recombinant T cells.

The method provided by the present invention is to knock out the BCOR gene and ZC3H12A gene of the target T cell or to inhibit the functions of the BCOR gene product and the ZC3H12A gene product of the target T cell, and to introduce a CAR structure or TCR structure or other corresponding structures of the adoptive cell transfer therapy with a target, to obtain a recombinant T cell.

The method provided by the present invention is to knock out the BCOR gene and ZC3H12A gene of the target T cell or to inhibit the functions, and to introduce a CAR structure or TCR structure or other corresponding structures of the adoptive cell transfer therapy with a target, and to introduce a carrier expressing related molecules for treating diseases, to obtain a recombinant T cell.

In the examples of the present invention, the corresponding molecules for treating diseases take the IL23R fusion protein for treating enteritis as an example.

The use of the above-mentioned recombinant T cells in the preparation of products for preventing and/or fighting tumors is also within the scope of protection of the present invention.

The use of the above-mentioned recombinant T cells in the preparation of products for inhibiting tumor growth and/or metastasis is also within the scope of protection of the present invention.

The use of the above-mentioned recombinant T cells in the preparation of products targeting cells for eliminating the CAR structure in the body is also within the scope of protection of the present invention.

The use of the above-mentioned recombinant T cells as drug carriers is also within the scope of protection of the present invention.

The present invention also provides a product for treating diseases, which is prepared according to the following method:
1) knocking out the BCOR gene and ZC3H12A gene of the target T cell or inhibiting the functions of the BCOR gene product and ZC3H12A gene product of the target T cell, and introducing a CAR structure or TCR structure or other corresponding structures of the adoptive cell transfer therapy with a target, and introducing a carrier expressing related molecules for treating diseases, to obtain a recombinant T cell;
2) introducing a carrier expressing related molecules for treating diseases into the recombinant T cell described in any one of claims 1-6, to obtain a recombinant T cell.

In the examples of the present invention, the corresponding molecules for treating diseases take the IL23R for treating enteritis and the GLP1 for treating obesity as examples.

The use of the substances for knocking out BCOR gene and ZC3H12A gene or substances for inhibiting the expressions of BCOR gene and ZC3H12A gene in any of the following is also within the scope of protection of the present invention:
1) Use in the preparation of products for promoting T cell expansion;
2) Use in the preparation of anti-tumor or anti-autoimmune disease products;
3) Use in the preparation of products for preventing tumors or preventing autoimmune diseases;
4) Use in the preparation of products for inhibiting tumor growth and/or metastasis;
5) Use in the preparation of recombinant T cells;
6) Use in the preparation of products for prolonging the persistence and/or stability of T cells in vivo;
7) Use in the preparation of T cells with memory effect;
8) Use in the preparation of T cells with self-renewal ability;
9) Use in the preparation of drug carriers for stable delivery of therapeutic biological formulations.

The experiments of the present invention proves that the CAR-T cells with knockout of double genes ZC3H12A/BCOR obtained by gene editing have great advantages compared with traditional CAR-T cells, that is, 1) no pretreatment with great toxic side effects is required for the patients; for example, in the tumor treatment, CAR-T treatment can be performed without chemotherapy pretreatment, and these CAR-T exist permanently in the body, achieving the purpose of curing tumors and preventing recurrence; in the mouse model, gene-edited CAR-T cells can massively expand in vivo and kill target cells without chemotherapy pretreatment; these cells have properties of stem cells and can exist indefinitely in vivo, achieving the purpose of cure; 2) only a small amount of cells are required to carry out the effective treatment; 3) gene-edited CAR-T cells persist in vivo, which is equivalent to stably implanting a group of cells in vivo for a long time, solving the problem of long-term effectiveness of CAR-T treatment, and a single treatment can achieve long-term treatment and prevention effects. 4) these CAR-T cells that persist in vivo can also be used as carriers to secrete proteins having therapeutic effects, including antibodies, polypeptides and hormones, etc. These cells can be used as a universal platform to secrete various therapeutic biological formulations (such as antibodies, polypeptides, hormones, etc.). This technology will greatly reduce the medical costs of repeated administration and achieve the purpose of curing some diseases.

### DETAILED DESCRIPTION OF THE INVENTION

### Recombinant Immune Cells

In one embodiment, the present invention provides a recombinant immune cell, in which the expression and/or functions of the BCOR gene and the ZC3H12A gene are reduced or eliminated.

In some embodiments, the immune cell is selected from one or more of T cells, B cells, NK cells, mast cells, and tumor-infiltrating lymphocytes, preferably T cells or NK cells; the T cell is selected from one or more of CD4+CD8+ T cells, CD8+ T cells, CD4+T cells, effector T cells, suppressor T cells, primitive T cells, memory T cells, γ-δT cells, α-βT cells, CD4-CD8-double negative T cells or NKT cells.

In some embodiments, the recombinant immune cell is a recombinant T cell, which does not comprise the BCOR gene and the ZC3H12A gene, or the biological functions of the BCOR gene product and the ZC3H12A gene product of the recombinant T cell are repressed.

TABLE 1
Target gene

| | | Human | Human | Mouse | Mouse |
|---|---|---|---|---|---|
| | | NCBI | Uniport | NCBI | Uniport |
| Gene | | Gene | reference | Gene | reference |
| symbol | Gene name | ID | number | ID | number |
| BCOR | BCL6 corepressor | 54880 | Q6W2J9 | 71458 | Q8CGN4 |
| ZC3H12A | Endoribonuclease | 80149 | Q5D1E8 | 230738 | Q5D1E7 |
| | ZC3H12A | | | | |

In some embodiments, the BCOR gene and ZC3H12A gene in the recombinant immune cell are treated with gene knockout technology, gene silencing technology, inactivation mutation technology, PROTAC technology or small molecule inhibitors.

In some embodiments, compared with unmodified or control immune cells, the expression or functions of the BCOR gene and ZC3H12A gene of the recombinant immune cell of the present invention is reduced by at least 50%, at least 60%, at least 70%, at least 80%, at least 90%, at least 95% or 100%, respectively.

In some embodiments, the recombinant immune cell of the present invention includes one or more structures for adoptive cell transfer therapy.

In some embodiments, the corresponding structure for adoptive cell transfer therapy is a chimeric antigen receptor (CAR) structure, a T cell antigen receptor (TCR) structure, a receptor structure based on ligand-receptor binding, or a synthetic T cell receptor and antigen receptor (STAR). For the description of STAR, see WO2020029774A1 and Yue Liu. et, al. Chimeric STAR receptors using TCR machinery mediate robust responses against solid tumors. Sci Transl Med. 2021 Mar. 24; 13 (586): eabb5191. doi: 10.1126/scitranslmed.abb5191.

In some embodiments, the antigen bound by the antigen receptor is selected from one or more of ROR1, Her2, L1-CAM, CD4, CD5, CD8, CD19, CD20, BCMA, CD7, Clauding 18.2, GPC3, MSLN, AFP, CD22, mesothelin, CEA, hepatitis B surface antigen, antifolate receptor, CD23, CD24, CD30, CD33, CD38, CD44, EGFR, EGFRVIII, EGP-2, EGP-4, EPHa2, ErbB2, ErbB3, ErbB4, FBP, fetal acetylcholine receptor, GD2, GD3, HMWMAA, IL-22R-α, IL-13R-α2, kdr, κ light chain, Lewis Y, L1-cell adhesion molecule (CD171), MAGE-A1, mesothelin, MUC1, MUC16, PSCA, NKG2D ligand, NY-ESO-1, MART-1, gp100, tumor embryonic antigen, TAG72, VEGF-R2, carcinoembryonic antigen (CEA), prostate specific antigen, PSMA, estrogen receptor, progesterone receptor, ephrin B2, CD123, CS-1, c-Met, MAGE A3, CE7, Wilms tumor 1 (WT-1), cyclin A1 (CCNA1), interleukin 12 or other tumor-associated antigens.

In some embodiments, the recombinant immune cell is an immune cell derived from a mammal. The mammals include primates (such as humans, monkeys), cattle, sheep, goats, alpacas, horses, dogs, cats, rabbits, rats, mice, etc.

In some embodiments, the recombinant immune cell also comprises genes expressing biological molecules for treating diseases. The biological molecules expressed for treating diseases are selected from one or more of cytokines, hormones, growth factors, coagulation factors, blood-cell expressed, chemokines, co-stimulatory molecules, activation peptides, antibodies or antigen binding fragments thereof. Specifically, the biological molecules for treating diseases are selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, and GLP1.

In some embodiments, at least 1 week, 2 weeks, 3 weeks, 4 weeks, 1 month, 2 months, 3 months, 4 months, 5 months, 6 months, 9 months, 12 months, 18 months, 2 years, 5 years, 10 years, 20 years, 40 years after the administration to the subject, the recombinant immune cells can be detected in the peripheral blood of the subject. In some embodiments, the recombinant immune cells are immortal-like immune cells. Such immortal-like recombinant immune cells are non-tumor cells.

In some embodiments, at least 1 week, 2 weeks, 3 weeks, 4 weeks, 1 month, 2 months, 3 months, 4 months, 5 months, 6 months, 9 months, 12 months after the administration to the subject, the proportion of the recombinant immune cells in which the expression and/or functions of the BCOR gene and ZC3H12A gene are reduced or eliminated is not less than 20%, 30%, 40%, 50%, 60%, 70%, 80%, 90%, 95% relative to the total amount of immune cells of the same type.

In other embodiments, at least 1 week, 2 weeks, 3 weeks, 4 weeks, 1 month, 2 months, 3 months, 4 months, 5 months, 6 months, 9 months, 12 months after the administration to the subject, the proportion of the recombinant immune cells in which the expression and/or functions of the BCOR gene and ZC3H12A gene are reduced or eliminated relative to the total number of the peripheral blood cells is selected from 1%-35%, 3-30%, 3-20%; the specific value can be any value within the above numerical range, including but not limited to 1%, 3%, 5%, 10%, 15%, 20%, 25%, 30%, 35%.

In other embodiments, compared with unmodified immune effector cells, the recombinant immune effector cells of the present invention exhibit increased or prolonged cell viability. In such embodiments, the result is that after a given period of time, the number of modified immune effector cells present is increased compared with unmodified immune effector cells. For example, in some embodiments, the period of time for which the modified immune effector cells of the present invention maintain viability and last is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100 or more times longer than that of the unmodified immune cells.

In some embodiments, the production of the biological molecules for treating diseases (e.g., IL23R, TNF or IL-5, GLP1) of the recombinant immune effector cells is increased by 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more times than the production of the biological molecules for treating diseases observed in the unmodified immune cell population.

### Preparation Method of Recombinant Immune Cells

In another embodiment, the present invention provides a preparation method of the above-mentioned recombinant immune cells. The method includes treating the BCOR gene and the ZC3H12A gene in the recombinant immune cells with gene knockout technology, gene silencing technology, or inactivation mutation technology, or small molecule inhibitors.

In some embodiments, the gene knockout technology comprises CRISPR/Cas technology, artificial zinc finger nucleases (ZFN) technology, transcription activator-like effector (TALE) technology, or TALE-CRISPR/Cas technology.

In some embodiments, the CRISPR/Cas technology is selected from the group consisting of CRISPR-Cas3, CRISPR-Cas9, CRISPR-Cas12, CRISPR-Cas13, CRISPR-CasX, or CRISPR-IscB system. For a description of the CRISPR-CasX system, see Liu J. J. et al., Nature, 2019 or https://doi.org/10.1016/j.molcel.2022.02.002. For a description of the CRISPR-IscB system, see Han Altae-Tran. et al., Science 374, Vol 374, Issue 6563, 57-65 (2021). DOI: 10.1126/science.abj6856.

In some embodiments, the CRISPR/Cas technology is specifically selected from the group consisting of CRISPR-Cas9, CRISPR-Cas12a, CRISPR-Cas12b, CRISPR-Cas13a, CRISPR-Cas13b, CRISPR-Cas13c, CRISPR-Cas13e or CRISPR-Cas13f system.

In some embodiments, the CRISPR/Cas technology uses a guide RNA (gRNA) targeting BCOR gene and Cas endonuclease, and guide RNA (gRNA) targeting ZC3H12A gene and Cas endonuclease.

In some embodiments, the guide RNA (gRNA) of the CRISPR/Cas technology simultaneously or respectively includes a guide RNA (gRNA) targeting BCOR gene and a guide RNA (gRNA) targeting ZC3H12A gene.

The present invention provides a guide RNA (gRNA) that directs a site-directed modified polypeptide to a specific target nucleic acid sequence. The gRNA comprises a nucleic acid targeting segment and a protein binding segment. The nucleic acid targeting segment of the gRNA comprises a nucleotide sequence that is complementary to a sequence in a target nucleic acid sequence. Therefore, the nucleic acid targeting segment of the gRNA interacts with the target nucleic acid in a sequence-specific manner via hybridization (i.e., base pairing), and the nucleotide sequence of the nucleic acid targeting segment determines the position within the target nucleic acid where the gRNA will bind. The nucleic acid targeting segment of the gRNA can be modified (e.g., by genetic engineering) to hybridize with any desired sequence within the target nucleic acid sequence.

The protein binding segment of the guide RNA interacts with the site-directed modified polypeptide (e.g., Cas protein) to form a complex. The guide RNA guides the bound polypeptide to a specific nucleotide sequence within the target nucleic acid through the above-mentioned nucleic acid targeting segment. The protein binding segment of the guide RNA comprises two nucleotide fragments that are complementary to each other and form a double-stranded RNA duplex.

In some embodiments, gRNA comprises two separate RNA molecules. In such embodiments, each of the two RNA molecules comprises a stretch of nucleotides that are complementary to each other, such that the complementary nucleotides of the two RNA molecules hybridize to form a double-stranded RNA duplex of the protein binding segment. In some embodiments, gRNA comprises a single RNA molecule (sgRNA).

The specificity of the gRNA for the target locus is mediated by the sequence of the nucleic acid binding segment, the nucleic acid binding segment comprises 20 nucleotides complementary to the target nucleic acid sequence within the target locus. In some embodiments, the length of the corresponding target nucleic acid sequence is 20 nucleotides. In some embodiments, the nucleic acid binding segment of the gRNA sequence of the present invention is at least 90% complementary to the target nucleic acid sequence within the target locus. In some embodiments, the nucleic acid binding segment of the gRNA sequence of the present invention is at least 95%, 96%, 97%, 98% or 99% complementary to the target nucleic acid sequence within the target locus. In some embodiments, the nucleic acid binding segment of the gRNA sequence of the present invention is 100% complementary to the target nucleic acid sequence within the target locus. In some embodiments, the target nucleic acid sequence is an RNA target sequence. In some embodiments, the target nucleic acid sequence is a DNA target sequence.

In some embodiments, the target nucleic acid sequence within the target locus must be changed. For example, the target nucleic acid sequence may change because the Cas protein used changes and the new Cas protein has a different PAM. The present description provides many examples of the target nucleic acid sequences of gRNA in the description and tables provided herein. Any of these target nucleic acid sequences can be altered by moving the target nucleic acid sequence at 5′ or 3′ within the target locus within a given gene. In some embodiments, the target nucleic acid sequence is moved up to 100 bp at 5′ or 3′ within the target locus within a given gene. In other embodiments, the target nucleic acid sequence is moved up to 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 bp at 5′ or 3′ within the target locus within a given gene (e.g., the BCOR gene and/or ZC3H12A gene of a human or mouse as described in Table 1).

In some embodiments, the nucleic acid binding segment in the guide RNA (gRNA) targeting the BCOR gene binds to a target DNA sequence having at least 90%, 95%, 96%, 97%, 98%, 99% or 100% identity with the DNA sequence encoded by the BCOR gene of a subject (e.g., NCBI Gene ID: 54880 or NCBI Gene ID: 71458); the nucleic acid binding segment in the guide RNA (gRNA) targeting the ZC3H12A gene binds to a target DNA sequence having at least 90%, 95%, 96%, 97%, 98%, 99% or 100% identity with the DNA sequence encoded by the ZC3H12A gene of a subject (e.g., NCBI Gene ID: 80149 or NCBI Gene ID: 230738).

In some embodiments, the nucleic acid binding segment in the guide RNA (gRNA) targeting the ZC3H12A gene binds to a target DNA sequence having at least 95%, 96%, 97%, 98%, 99% or 100% identity with the DNA sequence defined by a set of genomic coordinates shown in Table 7 or Table 8 of WO2020163365A2. Alternatively, the nucleic acid binding segment of the gRNA molecule targeting ZC3H12A binds to a target DNA sequence having at least 95%, 96%, 97%, 98%, 99% or 100% identity with one of the sequences shown in Tables 16 and 17 of WO2020163365A2.

In some embodiments, the targeting domain in the guide RNA (gRNA) targeting the BCOR gene comprises the sequence ACTGGGCAATACCGCAACAG (SEQ ID NO: 3) or a sequence having at least 85%, 90%, or 95% identity with SEQ ID NO: 3; the targeting domain of the guide RNA (gRNA) targeting the ZC3H12A gene comprises the sequence CTAGGGGAATTGGTGAAGCA (SEQ ID NO: 4) or a sequence having at least 85%, 90%, or 95% identity with SEQ ID NO: 4.

In some embodiments, the sequence of a CAR structure or TCR structure or other corresponding structures of the adoptive cell transfer therapy with a target is further introduced further into the immune cell.

In some embodiments, biological molecules expressed for treating diseases are further introduced into the immune cell; preferably, the biological molecules for treating diseases are selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, and GLP1.

In some embodiments, the carrier used is a viral vector, a virus-like vector, or a non-viral vector. In some embodiments, the recombinant vector comprising a polynucleotide encoding one or more components of the gene regulation system described herein is a viral vector. Suitable viral vectors include, but are not limited to the viral vectors based on the following: vaccinia virus, poliovirus, adenovirus, adeno-associated virus, SV40, herpes simplex virus, human immunodeficiency virus, retroviral vector (e.g., murine leukemia virus, spleen necrosis virus, and vectors derived from retroviruses, such as Rous sarcoma virus, Harvey sarcoma virus, avian leukosis virus, lentivirus, human immunodeficiency virus, myeloproliferative sarcoma virus, and mammary tumor virus), etc. Suitable non-viral vectors are selected from transposons, lipid nanoparticles, liposomes, exosomes, attenuated bacteria, or virus-like particles.

In some embodiments, the polynucleotide sequence encoding one or more components of the gene regulation system described herein is operably linked to a control element, such as a transcriptional control element, such as a promoter. The transcriptional control element may be functional in a eukaryotic cell (e.g., a mammalian cell) or a prokaryotic cell (e.g., a bacterial or archaeal cell). In some embodiments, the polynucleotide sequence encoding one or more components of the gene regulation system described herein is operably linked to multiple control elements, which allows the polynucleotide to express in both prokaryotic and eukaryotic cells. According to the cell type and gene regulation system used, any one of many suitable transcriptional and translational control elements (including constitutive and inducible promoters, transcriptional enhancer elements, transcriptional terminators, etc.) can be used in the expression vectors.

In some embodiments, non-limiting examples of suitable eukaryotic promoters (promoters that function in eukaryotic cells) include those from cytomegalovirus (CMV) immediate early, herpes simplex virus (HSV) thymidine kinase, early and late SV40, long terminal repeats (LTRs) from retroviruses, and mouse metallothionein 1. The selection of suitable carriers and promoters is well within the capabilities of those of ordinary skill in the art. The expression vector may also comprise a ribosome binding site for translation initiation and a transcriptional terminator. The expression vector may also comprise proper sequences for expanding expressions. The expression vector may also comprise a nucleotide sequence encoding a protein tag (e.g., 1c tag, hemagglutinin tag, green fluorescent protein, etc.) fused to the site-directed modified polypeptide so as to produce a chimeric polypeptide.

In some embodiments, the sgRNA expression vectors used include: the fundamental structure of vector-promoter 1-sgZc3h12a-promoter 2-tag-P2A-the biological molecule sequence for treating diseases, vector-promoter 1-sgBcor-promoter 2-tag-P2A-the biological molecule sequence for treating diseases or pMSCV-promoter 1-sgBcor-promoter 2-sgZc3h12a-promoter 3-tag-P2A-the biological molecule sequence for treating diseases. The above “-” does not represent a limitation on a specific connection sequence, and should be understood as an expression vector comprising related elements. The above-mentioned biological molecule sequence for treating diseases includes one or more of the structure sequence of the adoptive cell transfer therapy or the biological molecules for treating diseases in the recombinant immune cells of the present invention. Specifically, the sgRNA expression vector comprises the fundamental structure of pMSCV-hU6-sgZc3h12a-EFS-Thy1.1-P2A-CD19-CAR, pMSCV-hU6-sgBcor-EFS-Thy1.1-P2A-CD19-CAR or pMSCV-hU6-sgBcor-hU6-sgZc3h12a-EFS-Thy1.1-P2A-CD19-CAR. Further, each promoter in the expression vector, such as promoter 1, promoter 2 and promoter 3, can be the same or different; the tag is optionally present or absent; the biological molecule sequence for treating diseases is optionally present or absent.

In some embodiments, the method of preparing a recombinant immune cell of the present invention includes the step of introducing an expression vector into the recombinant immune cell. The methods for introducing polynucleotides and recombinant expression vectors into host cells are known in the art, and any known method can be used to introduce components of the gene regulation system into a cell. Suitable methods include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, microfluidic delivery methods, etc. In addition, cells can also be introduced by administration of non-viral delivery vehicles, the non-viral delivery vehicles may be such as transposons, nanoparticles (e.g., lipid nanoparticles), liposomes, exosomes, attenuated bacteria or virus-like particles.

### Gene Regulation System

In another embodiment, the present invention provides a gene regulation system. The gene regulation system is used for the preparation of the recombinant immune cells of the present invention.

In some embodiments, the gene regulation system of the present invention reduces the expression or function of the BCOR gene and the ZC3H12A gene in the immune cells by at least 50%, at least 60%, at least 70%, at least 80%, at least 90%, at least 95% or 100%, respectively.

In some embodiments, the gene regulation system of the present invention uses gene knockout technology, gene silencing technology or inactivation mutation technology or small molecule inhibitors to treat the BCOR gene and the ZC3H12A gene in the recombinant immune cells.

In some embodiments, the gene knockout technologies used include CRISPR/Cas technology, artificial zinc finger nuclease (Zinc Finger Nucleases, ZFN) technology, transcription activator-like effector (transcription activator-like effector, TALE) technology or TALE-CRISPR/Cas technology.

In some embodiments, the gene regulation system comprises a nucleic acid molecule and an enzyme protein, wherein the nucleic acid molecule is a guide RNA (gRNA) molecule, and the enzyme protein is a Cas protein or a Cas ortholog.

In some embodiments, the enzyme protein is selected from Cas9, Cas12a, Cas12b, Cas13a, Cas13b, Cas13c, Cas13e or Cas13f protein or the orthologs thereof.

In some embodiments, the gene regulation system of the present invention includes:
(i) the sequence of the targeting domain in the guide RNA (gRNA) targeting the BCOR gene is complexed with the first Cas endonuclease protein to form a first ribonucleoprotein (RNP) complex; and;
(ii) the sequence of the targeting domain in the guide RNA (gRNA) targeting the ZC3H12A gene is complexed with the second Cas endonuclease protein to form a second ribonucleoprotein (RNP) complex.

In some embodiments, the first ribonucleoprotein (RNP) complex and the second ribonucleoprotein (RNP) complex can be introduced into immune cells simultaneously, sequentially or successively.

In some embodiments, in the gene regulation system of the present invention, the nucleic acid binding segment in the guide RNA (gRNA) targeting the BCOR gene binds to a target DNA sequence having at least 90%, 95%, 96%, 97%, 98%, 99% or 100% identity with the DNA sequence encoded by the BCOR gene of the subject (e.g., NCBI Gene ID: 54880 or NCBI Gene ID: 71458); the nucleic acid binding segment in the guide RNA (gRNA) targeting the ZC3H12A gene binds to a target DNA sequence having at least 90%, 95%, 96%, 97%, 98%, 99% or 100% identity with the DNA sequence encoded by the ZC3H12A gene of the subject (e.g., NCBI Gene ID: 80149 or NCBI Gene ID: 230738).

In some embodiments, in the gene regulation system of the present invention, the targeting domain in the guide RNA (gRNA) targeting the BCOR gene comprises the sequence ACTGGGCAATACCGCAACAG (SEQ ID NO: 3) or a sequence having at least 85%, 90%, 95% identity with SEQ ID NO: 3; the targeting domain of the guide RNA (gRNA) targeting the ZC3H12A gene comprises the sequence CTAGGGGAATTGGTGAAGCA (SEQ ID NO: 4) or a sequence having at least 85%, 90%, 95% identity with SEQ ID NO: 4.

In some embodiments, in the gene regulation system of the present invention, the sequence of a CAR structure, a TCR structure, a receptor structure based on ligand-receptor binding, a STAR structure or other corresponding structures for adoptive cell transfer therapy with a target is further introduced into immune cells.

In some embodiments, in the gene regulation system of the present invention, genes are further introduced into immune cells for expressing biological molecules for treating diseases; preferably, the biological molecules for treating diseases are selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, and GLP1.

In some embodiments, the carrier used in the gene regulation system of the present invention is a viral vector, a virus-like vector or a non-viral vector. The viral vector is preferably a vaccinia virus, a poliovirus, an adenovirus, an adeno-associated virus, SV40, a herpes simplex virus, a human immunodeficiency virus, or a retroviral vector. The non-viral vector is preferably a transposon, a lipid nanoparticle, a liposome, an exosome, an attenuated bacterium or a virus-like particle.

In some embodiments, the RNA (gRNA) expression vector used in the gene regulation system of the present invention includes: the fundamental structure of vector-promoter 1-sgZc3h12a-promoter 2-tag-P2A-the sequence of the biological molecule for treating diseases, vector-promoter 1-sgBcor-promoter 2-tag-P2A-the sequence of the biological molecule for treating diseases or pMSCV-promoter 1-sgBcor-promoter 2-sgZc3h12a-promoter 3-tag-P2A-the sequence of the biological molecule for treating diseases. The above “-” does not represent a limitation on a specific connection order, and should be understood as an expression vector comprising related elements. The above-mentioned sequence of the biological molecule for treating diseases includes one or more of the structural sequences of the adoptive cell transfer therapy or the biological molecules for treating diseases in the recombinant immune cells of the present invention. Specifically, the sgRNA expression vector includes the fundamental structure of pMSCV-hU6-sgZc3h12a-EFS-Thy1.1-P2A-CD19-CAR, pMSCV-hU6-sgBcor-EFS-Thy1.1-P2A-CD19-CAR or pMSCV-hU6-sgBcor-hU6-sgZc3h12a-EFS-Thy1.1-P2A-CD19-CAR. Furthermore, each promoter in the expression vector, such as promoter 1, promoter 2 and promoter 3, may be the same or different; the tag may be optionally present or absent; the sequence of the biological molecule for treating diseases may be optionally present or absent.

In another embodiment, the present invention provides a kit comprising the above-mentioned gene regulation system.

### Method for Producing Recombinant Immune Cells

In another embodiment, the present invention provides a method for producing the above-mentioned recombinant immune cells, comprising:
(1) obtaining immune cells;
(2) treating immune cells using the preparation method described in any of the above embodiments, or introducing the gene regulation system described in any of the above embodiments into immune cells, or treating immune cells using the kit described in any of the above embodiments;
(3) culturing immune cells, and reducing or eliminating the expression and/or function of the BCOR gene and the ZC3H12A gene in the immune cells.

In some embodiments, the recombinant immune cells obtained in step (3) are implanted into a subject for expansion, and the recombinant immune cells after expansion in vivo or body are obtained. The recombinant immune cells obtained after expansion from the first-generation subject can be used for autologous treatment of the subject or for allogeneic treatment of other subjects.

In some embodiments, the immune cells are autologous immune cells or allogeneic immune cells to the subject.

### Composition

In another embodiment, the present invention provides a composition for treating diseases. The term “composition” refers to a formulation of the gene regulation system or modified immune effector cell as described herein, which can be administered or delivered to a subject or cell. A “therapeutic composition” or “pharmaceutical composition” (used interchangeably herein) is a composition of a gene regulation system or modified recombinant immune effector cell, which can be administered to a subject to treat a specific disease or condition, or contacted with a cell to modify one or more target genes.

In some embodiments, the composition for treating diseases comprises the recombinant immune cell as described in any of the above embodiments.

In some embodiments, the composition for treating diseases comprises the gene regulation system as described in any of the above embodiments.

### Method for Treating Diseases or Conditions

In another embodiment, the present invention provides a method for treating diseases or conditions in a subject in need thereof. The method comprises administering to the subject the recombinant immune cells described in any of the above embodiments, or administering the composition described in any of the above embodiments, or treating immune cells of the subject using the gene regulation system described in any of the above embodiments.

In some embodiments, the disease or condition is cancer, tumor, autoimmune disease, infectious disease, inflammatory disease, metabolic disease, neurodegenerative disease, disease caused by exogenous CAR structure targeting cells, disease caused by exogenous TCR structure targeting cells.

In some embodiments, the cancer or tumor comprises one or more of the following: leukemia, lymphoma, chronic lymphocytic leukemia (CLL), acute lymphocytic leukemia (ALL), non-Hodgkin's lymphoma, acute myeloid leukemia, multiple myeloma, refractory follicular lymphoma, mantle cell lymphoma, indolent B-cell lymphoma, B-cell malignancies, colon cancer, lung cancer, liver cancer, breast cancer, prostate cancer, ovarian cancer, skin cancer, melanoma, bone cancer, and brain cancer, ovarian cancer, epithelial cancer, renal cell carcinoma, pancreatic cancer, Hodgkin's lymphoma, cervical cancer, colorectal cancer, glioblastoma, neuroblastoma, Ewing's sarcoma, medulloblastoma, osteosarcoma, synovial sarcoma, and/or mesothelioma; the autoimmune disease comprises one or more of the following: ankylosing spondylitis (AS), psoriasis (PS), celiac disease (CEL), systemic lupus erythematosus (SLE), common variable immunodeficiency (CVID), inflammatory bowel disease (IBD), ulcerative colitis (UC), type I diabetes (TID), juvenile idiopathic arthritis (JIA), Crohn's disease (CD), alopecia areata (AA), multiple sclerosis (MS), primary biliary cirrhosis (PBC), primary sclerosing cholangitis (PSC), rheumatoid arthritis (RA), Sjogren's syndrome (SJO), systemic sclerosis (SSC), spondyloarthropathies (SPA), vitiligo (VIT), asthma, or thyroiditis (AITD, THY, or TH).

### Pharmaceutical Use

In another embodiment, the present invention provides a use of preparing a drug.

In some embodiments, the present invention provides the recombinant immune cell described in any of the above embodiments, for use in the manufacture of a medicament for treating a disease or condition in a subject in need thereof.

In some embodiments, the present invention provides the composition described in any of the above embodiments, for use in the manufacture of a medicament for treating a disease or condition in a subject in need thereof.

In some embodiments, the present invention provides the immune cell of the subject treated with the gene regulation system described in any of the above embodiments, for use in the manufacture of a medicament for treating a disease or condition in a subject in need thereof.

### Use of the Delivery Carrier

In another embodiment, the present invention provides an use of a recombinant immune cell as a carrier for stably delivering biological molecules for treating diseases.

In some embodiments, the recombinant immune cell is the recombinant immune cell described in any of the above embodiments, or the immune cell of a subject treated with a gene regulation system described in any of the above embodiments; the biological molecules delivered for treating diseases are selected from any one or more of biological molecules delivered for treating diseases described in any of the above embodiments.

In some embodiments, the biological molecules for treating diseases are selected from: one or more of cytokines, hormones, growth factors, coagulation factors, blood cell-expressed, chemokines, co-stimulatory molecules, activation peptides, antibodies or antigen-binding fragments thereof. Specifically, the biological molecules for treating diseases are selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, and GLP1.

Use of Reducing or Eliminating the Expression and/or Function of Target Genes

In another embodiment, the present invention provides a use of reducing or eliminating the expression and/or function of BCOR gene and ZC3H12A gene in immune cells.

In some embodiments, the use includes increasing the stemness of immune cells, inhibiting the exhaustion of immune cells, promoting the expansion of immune cells, conferring memory to immune cells, prolonging the persistence of immune cells, and increasing the self-renewal ability of immune cells.

In some embodiments, the recombinant immune cell is the recombinant immune cell described in any of the above embodiments, or the immune cell of a subject treated with a gene regulation system described in any of the above embodiments.

In some embodiments, the use is for therapeutic purposes or non-therapeutic purposes. In some embodiments, the non-therapeutic purpose includes using the immune cell for the preparation of a protein that can be encoded by DNA or for the preparation of a therapeutic composition.

Preferably, the protein that can be encoded by DNA includes any one or more of biological molecules delivered for treating diseases described in any of the above embodiments.

### Animal Model

In another embodiment, the present invention provides a method for producing an animal model.

In some embodiments, the method for producing an animal model uses the preparation method described in any of the above embodiments to treat the immune cells of an animal, or introduces the gene regulation system described in any of the above embodiments into the immune cells of an animal, or uses the kit described in any of the above embodiments to treat the immune cells of an animal.

In another embodiment, the present invention provides an animal model produced by the above method.

In another embodiment, the present invention provides the use of the recombinant immune cell, gene regulation system or kit described in any of the above embodiments in the manufacture of an animal model.

### Definitions

“BCOR gene” and “Bcor gene” can be used interchangeably on the premise of no specific definitions. The “BCOR gene” is the BCOR gene of any target subject.

“ZC3H12A gene” and “Zc3h12a gene” can be used interchangeably on the premise of no specific definitions. The “ZC3H12A gene” is the “ZC3H12A gene” of any target subject.

“Stemness” is also known as “stem cell properties”, which refers to the ability of cells to self-renew and differentiate into different cells.

“Immortal-like and Functional” (abbreviated as IF) refers to the properties of cells that acquire the ability to continue to grow and proliferate, and have no phenotypic characteristics of malignant transformation, no tumorigenicity, and no invasiveness and metastasis of tumor cells. Herein, the subscript IF is used to represent T cells with simultaneous knockout of Bcor and Zc3h12a and with “immortal-like and functional” properties, which is short for T_{IF}, including CAR19T_{IF}, GD2T_{IF} and EGFRT_{IF}.

“Subject” or “host” refers to human or non-human animals, including mammals. For example, primates (such as humans, monkeys), cattle, sheep, goats, alpacas, horses, dogs, cats, rabbits, rats, mice, etc. “Subject” or “host” includes therapeutic and non-therapeutic types. “Subject” or “host” includes experimental animal models or animals used to produce and express biological molecules for treating diseases, that is, “non-therapeutic host” or “non-therapeutic subject”.

“Related molecules for treating diseases” or “biological molecules for treating diseases” refers to related molecules or biological molecules that are introduced into immune cells through “exogenous genes” and secreted by recombinant immune cells on the premise of no specific definitions.

### Beneficial Effects

The experiments of the present invention prove that the CAR-T cells with knockout of ZC3H12A/BCOR double gene obtained by gene editing in the present invention have great advantages compared with traditional CAR-T cells, these advantages cannot be obtained by knocking out ZC3H12A or BCOR gene alone:
1) No pretreatment with great toxic side effects is required for the patients; for example, in the tumor treatment, CAR-T treatment can be performed without chemotherapy pretreatment, and these CAR-T cells exist permanently in the body, achieving the purpose of curing tumors and preventing recurrence; in the mouse model, gene-edited CAR-T cells can expand massively in vivo and kill target cells without chemotherapy pretreatment; these cells have properties of stem cells and can exist indefinitely in vivo, achieving the purpose of cure;
2) Effective treatment can be carried out using a small amount of recombinant cells;
3) Gene-edited CAR-T cells persist in vivo, which is equivalent to stably implanting a group of cells in vivo for a long time, solving the problem of long-term effectiveness of CAR-T treatment, and a single treatment can achieve long-term treatment and prevention effects.
4) These CAR-T cells that persist in vivo can also be used as carriers to secrete proteins having therapeutic effects, including antibodies, polypeptides and hormones, etc. These recombinant immune cells can be used as a universal carrier platform to secrete various therapeutic biological molecules (such as antibodies, polypeptides, hormones, etc.). This technology will greatly reduce the medical costs of repeated administration and achieve the purpose of curing some diseases.

### BRIEF DESCRIPTION OF THE DRAWINGS

FIG. 1. Identification of recombinant CAR-T cells with knockout of Bcor and/or Zc3h12a. Wherein, a refers to the expression level of the protein encoded by the target gene detected by immunoblotting after knockout of the Zc3h12a gene in the recombinant CAR-T cells; b and c refer to the editing results of the target gene after PCR (b) and gene sequencing detection (c) after knockout of the Bcor gene in the recombinant CAR-T cells.

FIG. 2. CAR19T_{IF} can efficiently and persistently expand after being infused back into mice under the condition of no pretreatment; CAR19T cells with knockout of Zc3h12a alone cannot persist and cannot continuously kill CD19+ target cells; wherein, a is a schematic diagram of the experimental procedure, b refers to the proportions of mCD19CAR cells (i.e., Thy1.1+ cells) back-infused in the peripheral blood of mice in each group in total CD8T cells after 7 days and 2 months analyzed by flow cytometry; c refers to the statistical curve of the change in the proportions of mCD19CAR cells in the peripheral blood of mice in total CD8T cells analyzed by flow cytometry from the 1st week to the 8th week after each group of mCD19CAR cells were infused, for PBS group, n=3, for other groups, n=5-6; d refers to the proportions of mCD19CAR cells in each group in total spleen cells in the spleen of mice analyzed by flow cytometry 6 months after the back-infusion, for sgNT group, n=3, for CAR19T_{IF} group, n=5. Data are mean±SEM, using unpaired student's t-test: NS, no significant difference; *p<0.05; **p<0.01; ***p<0.001; ****p<0.0001.

FIG. 3. CAR19T_{IF} have the properties of stem cells, and can be repeatedly passaged in different batches of mice without exhaustion. Wherein, a is the experimental design of repeated passage and infusion of CAR19T_{IF} cells in B6 mice; b-d refer to the statistical analyses of the proportions and number of CAR19T_{IF} cells in the spleen; e refers to the statistical analysis of the proportion of CD19+B cells in the spleen, n=4-6. f refers to the survival time of CAR19 cells and CAR19T_{IF} cells cultured in vitro; g is the experimental design of repeated passage and infusion of high-replication CAR19T_{IF} cells in B6 mice; h and i refer to the statistical analyses of the proportion and number of CFSE-CAR19T_{IF} cells in the spleen. Data are mean±SEM, using unpaired student's t-test: NS, no significant difference; *p<0.05; **p<0.01; ****p<0.0001.

FIG. 4. A small amount (500) of CAR19T_{IF} can efficiently expand and eliminate all the target cells in vivo under the condition of no pretreatment, but CAR19T_{IF} are self-limiting and will not overproliferate. Wherein, a is the experimental design of infusion of CAR19T_{IF} cells with a quantitative gradient in B6 mice; b to d refer to statistical analyses of the proportion and number of CAR19T_{IF} cells in the spleen; e refers to the statistical analysis of the proportion of CD19+B cells in the spleen. n=3-5, data are mean±SEM, using unpaired student's t-test: NS, no significant difference; *p<0.05; **p<0.01; ****p<0.0001.

FIG. 5. CAR19T_{IF} have a therapeutic effect on primary tumors and prolong the survival period of tumor-bearing mice. Wherein, a is a schematic diagram of the experimental procedure, B6 mice are subcutaneously inoculated with 2×10⁵ MC38 tumor cells expressing mCD19 (MC38-mCD19), 10 days later, 3×10⁶ CAR19T_{IF} cells are infused through the tail vein of mice, and tumor growth and survival of tumor-bearing mice are monitored, n=4. b refers to the tumor size, data are mean±SEM, using unpaired student's t-test: ***p<0.001; c refers to the survival curve of tumor-bearing mice, using log-rank (Mantel-Cox) test: ** P<0.01.

FIG. 6. CAR19T_{IF} have an immune memory protective effect on tumors and can prevent tumor recurrence for a long time. Wherein, a-c refer to experiments on colon cancer MC38: a is a flow chart, CAR19T_{IF} cells are infused through the tail vein of mice, one month later, 5×10⁵ MC38-mCD19 tumor cells are subcutaneously inoculated in B6 mice, and tumor growth and survival of tumor-bearing mice are monitored; b refers to the tumor size, for PBS control group, n=5, for the CAR19T_{IF} pre-inoculation group, n=10, data are mean±SEM, using unpaired student's t-test: ***p<0.001; c refers to the survival curve of tumor-bearing mice, using log-rank (Mantel-Cox) test: ** P<0.01; d-f refer to experiments on melanoma B6F10-mCD19 model, CAR19T_{IF} cells are infused through the tail vein of mice, one month later, 1×10⁵ B6F10-mCD19 cells are transplanted through the tail vein of B6 mice, 3 weeks later, the lung tumor burden of mice is checked, and the survival of mice is monitored; d refers to a schematic diagram of the melanoma B6F10 experimental procedure; e refers to a photo of lung tumor burden; f refers to the survival curve of tumor-bearing mice, for control group, n=5, for pre-inoculation group, n=6, data are mean±SEM, using log-rank (Mantel-Cox) test: ** P<0.01.

FIG. 7. Construction and identification of hCAR19T_{IF} cells in humanized hCD19 mice. Wherein, a refers to a schematic diagram of the experimental design for continuous transfer of hCAR19T_{IF} cells in humanized CD19 (hCD19) mice; b refers to the proportion of hCAR19T_{IF} (Thy1.1+) and the proportion of CD19+B cells in the spleen of the first-generation and the second-generation recipient hCD19 mice infused with hCAR19T_{IF}; c and d refer to the statistical analyses of the proportion and absolute number of hCAR19T_{IF} in the spleen of the first-generation and the second-generation recipient hCD19 mice, n=3, data are mean±SEM, using unpaired student's t-test: NS, no significant difference; **p<0.01; ****p<0.0001.

FIG. 8. The therapeutic effect of CD19CART_{IF}-IL23R adoptive cell transfer therapy on dextran sulfate sodium salt (DSS in the figure)-induced enteritis. a and b refer to the expression of IL23R in the cell fragments (a) and cell supernatant (b) of retroviral packaging cells detected by the method of Western blot. The control lane is a cell protein sample transfected with an empty carrier; c refers to a schematic diagram of the construction of the mouse enteritis model and CAR19T_{IF}-IL23R adoptive cell transfer therapy; d refers to the effect of CART_{IF}-IL23R adoptive cell transfer therapy on the body weight of mice with enteritis induced by dextran sulfate sodium salt (DSS in the figure). n=5, data are mean±SEM, using unpaired student's t-test: NS, no significant difference; *p<0.05; ***p<0.0001.

FIG. 9. GD2T_{IF} cells induced by simultaneous knockout of Bcor and Zc3h12a. Knockout of Bcor or Zc3h12a alone could not promote the expansion and persistence of GD2 CAR-T cells. Wherein, a refers to the CAR-T structure and the gene knockout carrier structure; b refers to the experimental flow chart; c refers to the representative flow cytometry plot; d refers to the statistical analysis, n=3, data are mean±SEM, using unpaired student's t-test: NS, no significant difference; **p<0.01.

FIG. 10. GD2T_{IF} cells have properties of stem cells and can be passaged in B6 mice and NSG mice while retaining T cell functions; but they will not form tumors in mice and possess safety. Wherein, a refers to the experimental flow chart; b and e refer to the representative flow cytometry plots; h refers to the survival time of GD2T_{IF} cells in vitro; i refers to the phenotypic analysis of GD2T_{IF} cells; k refers to the analysis of IFN□ production in GD2T_{IF} cells; c, d, f, g, j, 1, m refer to statistical analyses, n=3-6, data are mean±SEM, using unpaired student's t-test: NS, no significant difference; *p<0.05, **p<0.01, ***p<0.001, ****p<0.0001.

FIG. 11. EGFRT_{IF} cells induced by simultaneous knockout of Bcor and Zc3h12a. Knockout of Bcor or Zc3h12a alone cannot promote the expansion and persistence of EGFR CAR-T cells. Wherein, a refers to the CAR-T structure and gene knockout carrier structure; b refers to the experimental flow chart; c refers to the representative flow cytometry plot; d refers to the statistical analysis, n=3, data are mean±SEM, using unpaired student's t-test: ***p<0.001.

FIG. 12. EGFRT_{IF} cells have properties of stem cells and can be passaged in B6 mice while retaining T cell functions; but they will not form tumors in mice and possess safety. Wherein, a refers to the experimental flow chart; b refers to the representative flow cytometry plot; c and d refer to statistical analyses, n=6, data are mean±SEM, using unpaired student's t-test: ****p<0.0001; e refer to the survival time of GD2T_{IF} cells in vitro.

FIG. 13. EGFRT_{IF} cells inhibit the growth of colon cancer CT26 tumors under the condition that the tumor-bearing mice are not pretreated. Wherein, a refers to the schematic diagram of the experimental procedure, B6 mice are subcutaneously inoculated with 2×10⁵ MC38 tumor cells expressing mCD19 (MC38-mCD19). Three days later, 1×10⁶ CAR19T_{IF} cells are infused through the tail vein of mice, and tumor growth and survival of tumor-bearing mice are monitored, n=5. b refers to the tumor size, data are mean±SEM, using unpaired student's t-test: ***p<0.001.

FIG. 14. GD2T_{IF} cells are used as carriers to continuously secrete TNF to induce chronic inflammation model. Wherein, a refers to the schematic diagram of the principle; b refers to the representative flow cytometry plot; c and d refer to statistical analyses, n=4, data are mean±SEM, using unpaired student's t-test: ****p<0.0001; e refers to the change in body weight.

FIG. 15. GD2T_{IF} cells are used as carriers to continuously secrete IL-5 to induce eosinophilia model. Wherein, a refers to the schematic diagram of the principle; b refers to the representative flow cytometry plot; c and d refer to statistical analyses, n=4, data are mean±SEM, using unpaired student's t-test: ****p<0.0001.

FIG. 16. GD2T_{IF} cells are used as carriers to continuously secrete GLP1 to treat obesity and diabetes. Wherein, a refers to the schematic diagram of the principle; b and c refer to the weight change chart of the mice after treatment, n=5, data are mean±SEM, using unpaired student's t-test: ****p<0.0001, NS, no significant difference.

### DETAILED EMBODIMENTS

Unless otherwise specified, the experimental methods used in the following examples are all conventional methods.

Unless otherwise specified, the materials, reagents, etc. used in the following examples are all available from commercial sources.

### Example 1. Preparation of Recombinant CAR-T Cells with Knockout of Bcor and/or Zc3h12a

### 1. Construction of Gene Knockout Carrier

The present example constructed retrovirus-based sgRNA expression vectors, namely pMSCV-hU6-sgNT-EFS-Thy1.1-P2A-CD19-CAR, pMSCV-hU6-sgBcor-EFS-Thy1.1-P2A-CD19-CAR, pMSCV-hU6-sgZc3h12a-EFS-Thy1.1-P2A-CD19-CAR and pMSCV-hU6-sgBcor-hU6-sgZc3h12a-EFS-Thy1.1-P2A-CD19-CAR;
Wherein:
Carrier pMSCV-hU6-sgNT-EFS-Thy1.1-P2A-CD19-CAR (SEQ ID NO: 1), wherein position 257-276 was the random sequence SEQ ID NO: 2 that did not target any gene, serving as a control for no gene knockout;
Carrier pMSCV-hU6-sgBcor-EFS-Thy1.1-P2A-CD19-CAR (the carrier sequence was obtained by replacing position 257-276 of SEQ ID NO: 1 with SEQ ID NO: 3, while keeping other sequences unchanged), SEQ ID NO: 3 was the target sequence recognition region of sgBcor for knocking out Bcor, used for knocking out Bcor;
Carrier pMSCV-hU6-sgZc3h12a-EFS-Thy1.1-P2A-CD19-CAR (the carrier sequence was obtained by replacing position 257-276 of SEQ ID NO: 1 with SEQ ID NO:4, and keep other sequences unchanged), SEQ ID NO:4 was the target sequence recognition region of sgZc3h12a for knocking out Zc3h12a, used for knocking out Zc3h12a;
pMSCV-hU6-sgBcor-hU6-sgZc3h12a-EFS-Thy1.1-P2A-CD19-CAR (SEQ ID NO:5), wherein position 242-261 was the target sequence recognition region of sgBcor in mice for knocking out Bcor (SEQ ID NO:3), position 687-706 was the target sequence recognition region of sgZc3h12a for knocking out Zc3h12a in mice (SEQ ID NO:4), used for knocking out Bcor in mice and Zc3h12 in mice at the same time, all carriers were obtained by whole gene synthesis.

The sgRNA adopted above was shown in Table 2 below:

TABLE 2
the target sequence recognition region of sgRNA

| | |
|---|---|
| sgNon-targeting (sgNT) | TTCGCACGATTGCACCTTGG (SEQ ID NO: 2, |
| | corresponding to position 257-276 of SEQ ID NO: 1) |
| sgBcor | ACTGGGCAATACCGCAACAG (SEQ ID NO: 3) |
| sgZc3h12a | CTAGGGGAATTGGTGAAGCA (SEQ ID NO: 4) |

### 2. Isolation and Activation of Naive CD8 T Cells

Naive CD8 T cells were isolated and obtained from the spleen of Cas9 transgenic mice (from Jaxson Laboratory, #026430) by the magnetic bead sorting method. The cells were inoculated at a density of 10⁶ cells/well into a 12-well cell culture dish coated with the amount of 1 μg/ml anti-CD3 antibody (CD38, BioXcell #BE0001-1), and 2 ml RPMI1640 culture medium (containing 5% fetal bovine serum and interleukin-2) was added, at the same time, the amount of 1 μg/ml anti-CD28 antibody (BioXcell #BE0015-1) was added for in vitro activation, that is, the cells were cultured in an incubator with 5% carbon dioxide and 37° C., and infected with the virus after 36 hours of culture.

3. Construction of mCD19CAR Cells with Knockout of Bcor and Zc3h12a

### 1) Preparation of Retrovirus

After 24 hours of adherent culture, 10⁶ Phoenix-Eco cells (ATCC #CRL-3214) were co-transfected with the amount of 20 μg of the sgRNA expression vector pMSCV-hU6-sgBcor-hU6-sgZc3h12a-EFS-Thy1.1-P2A-CD19-CAR prepared in the above section 1 and the amount of 60 μg of the packaging plasmid pCL-Eco (purchased from Addgene #12371) by calcium phosphate precipitation method. After 48 hours of transfection, the supernatant containing the packaged virus was harvested, the virus supernatants were filtered through a 0.45 μm filter membrane to remove dead cell impurities to obtain the retrovirus supernatant, i.e., the retrovirus with knockout of Bcor and Zc3h12a.

### 2) Retroviral Infection

The amount of 1×10⁶ CD8 T cells after 36 hours of culture of in vitro culture and activation in step 2 was added to 1 ml of the retroviral supernatant obtained in step 1) and mixed, and then centrifuged at room temperature at the level of 2000 g for 2 hours. Then, it was placed in a carbon dioxide incubator and cultured for 4 hours, replaced with 2 ml of fresh RPMI1640 culture medium (containing 5% fetal bovine serum and the amount of 2 ng/ml of interleukin-2) and continued to be cultured (this time was recorded as the post-infection time). Thy1.1-positive cells (Thy1.1-biotin, BioLegend #202510) were sorted by using flow cytometry, that is, mCD19CAR cells with simultaneous knockout of Bcor and Zc3h12a (expressed as sgBcor/Zc3h12a) were obtained, which were named as CAR19T_{IF}, wherein IF stands for Immortal-like and Functional, and the Chinese translation is “immortal-like T cells”.

4. Construction of CD8 T Cells with Knockout of Zc3h12a

The only difference from “3. Construction of mCD19CAR cells with knockout of Bcor and Zc3h12a” was that “sgRNA expression vector pMSCV-hU6-sgBcor-hU6-sgZc3h12a-EFS-Thy 1.1-P2A-CD19-CAR” was replaced with “sgRNA expression vector pMSCV-hU6-sgZc3h12a-EFS-Thy1.1-P2A-CD19-CAR”, and other steps were kept unchanged, thus mCD19CAR cells with knockout of Zc3h12a were obtained (sgZc3h12a-mCD19CAR).

5. Construction of mCD19CAR Cells without Knocking Out Genes

The only difference from “3. Construction of mCD19CAR cells with knockout of Bcor and Zc3h12a” was that “sgRNA expression vector pMSCV-hU6-sgBcor-hU6-sgZc3h12a-EFS-Thy 1.1-P2A-CD19-CAR” was replaced with “sgRNA expression vector pMSCV-hU6-sgNT-EFS-Thy1.1-P2A-CD19-CAR”, and other steps were kept unchanged, thus mCD19CAR cells without knocking out genes were obtained (sgNT-mCD19CAR).

6. Identification of mCD19CAR Cells with Knockout of Bcor and Zc3h12a

### (1) Detect Knockout of Zc3h12a by Immunoblotting

Four days after CD8 T cells were infected with retroviruses that knocked out Bcor and Zc3h12a in the above section 3, Thy1.1-positive cells were sorted out by using flow cytometry, i.e., mCD19CAR cells with simultaneous knockout of sgBcor/Zc3h12a, cell lysates were prepared, the knockout effect of gene Zc3h12a was detected by adopting the conventional immunoblotting method (using an antibody that recognized Zc3h12a (purchased from Abcam, #ab211659)). The mCD19CAR cells without knocking out genes obtained in the above section 5 were taken as controls.

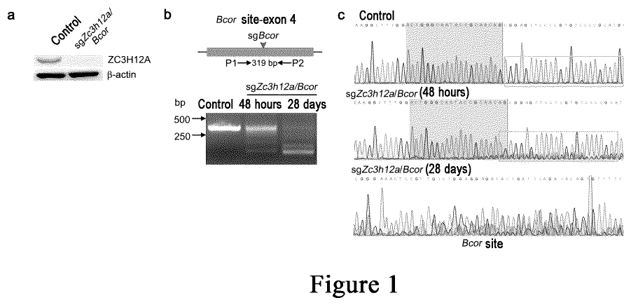

The results of 4 days after infection were shown in FIG. 1a, it can be seen that compared with mCD19CAR cells without knocking out any gene (control), the proteins encoded by the Zc3h12a gene in mCD19CAR cells with sgBcor/Zc3h12a knockout (represented as sgZc3h12a/Bcor in the figure) were completely knocked out.

### (2) PCR Identification of Bcor Knockout

The mCD19CAR cells with simultaneous knockout of sgBcor/Zc3h12a obtained 48 hours after CD8 T cells were infected with retroviruses with Bcor and Zc3h12a knockout in the above section 3 were taken and 28 days after these cells were infused back to mice, the mCD19CAR cells with simultaneous knockout of sgBcor/Zc3h12a were isolated and obtained again from the spleens of mice, and DNA was extracted respectively as a template for PCR amplification using the following primers. The mCD19CAR cells without knocking out genes obtained in the above section 5 were taken as the control. The primer sequence P1 for the identification of the gene editing of Bcor in mice was SEQ ID NO: NO: 6 (CCGAAAGAAACACTATCTCC), the primer sequence P2 for the identification of the gene editing of Bcor in mice was SEQ ID NO:7 (TGATGGCGTGGTATCCACCG).

The results were shown in FIG. 1b. The upper figure shows the positions of the primers, and the lower figure shows the PCR amplification results; The control was sgNT-mCD19CAR, 48 hours refers to the mCD19CAR cells with simultaneous knockout of sgBcor/Zc3h12a obtained 48 hours after retroviral infection (represented as sgZc3h12a/Bcor in the figure), and 28 days refers to the mCD19CAR cells with simultaneous knockout of sgBcor/Zc3h12a isolated and obtained from the spleens 28 days after the mice were back-infused 48 hours after retroviral infection (represented as sgZc3h12a/Bcor in the figure); It can be seen that compared with mCD19CAR cells without knocking out any genes (control), bands with normal size of Bcor gene cannot be detected for the Bcor gene in mCD19CAR cells with sgBcor/Zc3h12a knockout, indicating that the Bcor gene was successfully edited.

Sequencing was performed on the above PCR products, the results were shown in FIG. 1c. It can be seen that the corresponding sites of the Bcor and Zc3h12a genes had already been successfully edited.

### Example 2: MCD19 CAR-T Cells that were Back-Infused with Simultaneous Knockout of Zc3h12a and Bcor Genes and in the Case of No Pretreatment Expanded Efficiently, Persisted In Vivo, and Persistently Killed CD19+ Target Cells; mCD19 CAR-T Cells that Knocked Out Zc3h12a Gene Alone could not Persist, Nor could they Continuously Kill CD19+ Target Cells

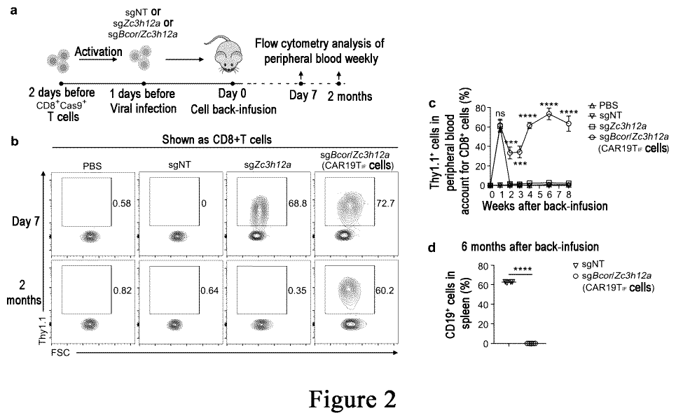

The cell reinfusion process was shown in FIG. 2a: CD8 T cells were isolated from the spleen and lymph nodes of Cas9 transgenic mice, and activated via CD3/CD28 for 24 hours to obtain activated CD8 T cells (the method was the same as that of section 2, Example 1); then the retrovirus obtained by transfection with pMSCV-hU6-sgNT-EFS-Thy1.1-P2A-CD19-CAR, the retrovirus obtained by transfection with pMSCV-hU6-sgZc3h12a-EFS-Thy1.1-P2A-CD19-CAR and the retrovirus obtained by transfection with pMSCV-hU6-sgBcor-hU6-sgZc3h12a-EFS-Thy1.1-P2A-CD19-CAR were used to infect the activated CD8 T cells respectively, the obtained recombinant cells were named as sgNT-mCD19CAR, sgZc3h12a-mCD19CAR and CAR19T_{IF} cells (sgZc3h12a/sgBcor cells) (the method was the same as that of section 3, Example 1), and the above cells obtained 24 hours after infection were respectively injected into C57bl/B6 mice (hereinafter referred to as B6 mice) through the tail vein of the mice, and the specific injection method was as follows:

6-8 week-old B6 mice weighing 20-25 g were divided into three groups, namely control (sgNT) group (4 mice), sgZc3h12a group (4 mice) and sgBcor/Zc3h12a group (4 mice).

Control (sgNT) group: The mCD19CAR cells without knocking out genes prepared by the method of Example 1 were formulated into a cell suspension with PBS, and infused back into each mouse in the sgNT group through the mice tail. Each mouse was back-infused into 4×10⁵ mCD19CAR cells without knocking out genes through their tails;

sgZc3h12a group: The mCD19CAR cells with knockout of Zc3h12a prepared by the method of Example 1 were formulated into a cell suspension with PBS, and infused back into each mouse in the sgZc3h12a group through the mice tail. Each mouse was back-infused into 4×10⁵ mCD19CAR cells with knockout of Zc3h12a gene through their tails;

sgBcor/Zc3h12a group (named as CAR19T_{IF}): The mCD19CAR cells with knockout of Bcor and Zc3h12a prepared by the method in Example 1 were formulated into a cell suspension with PBS, and infused back into each mouse in the sgBcor/Zc3h12a group through the mice tail. Each mouse was back-infused into 4×10⁵ mCD19CAR cells with knockout of Bcor and Zc3h12a genes through their tails.

The T cells were not back-infused in the PBS group, an equal volume of PBS was infused as a treatment control.

On the 7th day and 2nd month after the back-infusion respectively, the proportions of mCD19CAR cells without knocking out genes, mCD19CAR cells with knockout of Bcor, mCD19CAR cells with knockout of Zc3h12a, and mCD19CAR cells with simultaneous knockout of Bcor and Zc3h12a in the total CD8 T cells back-infused in the peripheral blood of each mouse were analyzed by flow cytometry weekly using the Thy1.1 antibody (with Thy1.1 screening tag on the knockout carrier).

The results were shown in FIG. 2b. In the control group (represented by “sgNT” in the figure), 4×10⁵ mCD19CAR cells without knocking out genes were infused back into the mice. On the 7th day and the 2nd month, these back-infused mCD19CAR cells accounted for only 0 and 0.64% of the total CD8 T cells in the peripheral blood. On the 7th day and the 2nd month, the mCD19CAR cells with knockout of Bcor in the sgZc3h12a group (represented by “sgZc3h12a” in the figure) accounted for 68.8% and 0.35% of the total CD8 T cells in the peripheral blood.

On the 7th day and the 2nd month, the mCD19CAR cells with simultaneous knockout of Bcor and Zc3h12a in the sgBcor/Zc3h12a group (represented by “sgBcor/Zc3h12a” in the figure) accounted for 72.7% and 60.2% of the total CD8 T cells in the peripheral blood.

The above results indicate that without any pretreatment on the mice, knockout of Zc3h12a can promote the efficient expansion of the back-infused mCD19CAR cells in normal B6 mice within 7 days, but they cannot proliferate at the time of 2 months, indicating poor persistence in vivo; while simultaneous knockout of Bcor and Zc3h12a significantly enhanced the expansion of mCD19CAR cells whether 7 days or 2 months after the back-infusion, with a significant synergistic effect, and the cells survived for a long time in vivo.

The changes in the proportions of back-infused mCD19CAR cells (Thy1.1+CD8 T cells) in total CD8 T cells in each group in the peripheral blood of mice analyzed by flow cytometry within 8 weeks after infusion back to mice were summarized respectively, the results were shown in FIG. 2c. PBS refers to the PBS control group, sgNT refers to the sgNT group, sgZc3h12a refers to the sgZc3h12a group, sgBcor/Zc3h12a refers to the sgBcor/Zc3h12a group. It can be seen that when Bcor and Zc3h12a were knocked out at the same time, the back-infusion time increased and the back-infused cells still expand significantly, indicating that the persistence of the cell survival in vivo was significantly higher than that of the back-infused cells in other groups.

At the time of six months after the back-infusion, spleen cells of the mice were obtained, the proportion of endogenous CD19+ cells (the antibody was CD19-biotin, BioLegend #101504) in total spleen cells in the spleen of the mice was detected by flow cytometry analysis, the results were shown in FIG. 2d, sgNT refers to the sgNT group, sgBcor/Zc3h12a refers to the sgBcor/Zc3h12a group. It can be seen that compared with sgNT, still no endogenous CD19-expressing B cells were detected in the spleen of the mice in sgBcor/Zc3h12a group 6 months after the back-infusion, indicating that the single back-infusion of CAR19T_{IF} can exert a long-term effect of eliminating targeted cells.

### Example 3: CAR19T_{IF} Produced by Simultaneous Knockout of Zc3h12a and Bcor Genes had Real Stem Cell Properties, and can be Repeatedly Passaged in Different Batches of Mice without Exhaustion; mCD19 CAR-T Cells with Knockout of Zc3h12a or Bcor Gene Alone Did not have Stem Cell Properties

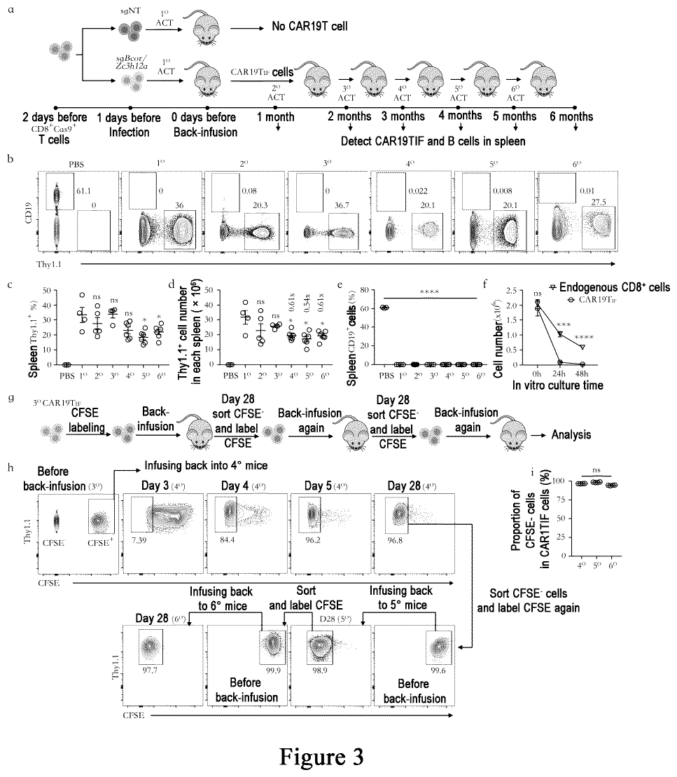

The method for preparing gene-knockout mCD19 CAR-T cells was the same as that in Example 2. The flow chart was shown in FIG. 3a. CAR19T_{IF} were taken out from the mice of the first generation, counted, and infused into the mice of the second generation through the tail vein (10⁶ cells were infused into each mouse). One month later, the above operations were repeated, the second-generation CAR19T_{IF} were infused into the third-generation mice, and this was repeated 6 times. At the time of one month after back-infused into the first-generation mice with wild-type mCD19 CAR-T cells and mCD19 CAR-T cells with knockout of Zc3h12a or Bcor gene alone, there were no mCD19 CAR-T cells in the spleen, thus the passage experiment could not be performed.

The results were shown in FIGS. 3b-3e. During the process of repeated passage, there was no obvious decrease in the proportion and number of CAR19T_{IF} in the spleen, and all CD19+B cells in each generation of mice were all internally eliminated. It should be noted here that real stem cells such as hematopoietic stem cells can only be passed for 3-4 generations under similar conditions. The experimental results indicated that CAR19T_{IF} had real stemness while retaining the killing function of mature T cells.

FIG. 3f showed that CAR19T_{IF} cells cannot survive in vitro, indicating that CAR19T_{IF} were not transformed into tumor cells.

As shown in FIG. 3g, during the passage process, CSFE was used in the present invention to label CAR19T_{IF} cells to detect whether the stemness of CAR19T_{IF} was mediated by a small group of slowly proliferating cells. In this experiment, the CSFE signal was inversely proportional to the number of cell divisions. The more cell divisions, the lower the CSFE cells. The results were shown in FIGS. 3h and 3i, CAR19T_{IF} that underwent multiple divisions in the mice of the previous generation could still massively expand in the mice of the next generation. Therefore, the stemness of CAR19T_{IF} cells was not mediated by a small group of cells, the entire CAR19T_{IF} cell population had the stemness.

### Example 4: A Small Amount (500) of CAR19T_{IF} Could Efficiently Expand and Eliminate all the Target Cells In Vivo Under the Condition of No Pretreatment, but CAR19T_{IF} were Self-Limiting and would not Overproliferate

One month after the back-infusion of the recipient mice infused back with CAR19T_{IF} cells in the above Example 2, spleen cells of the recipient mice were taken, and Thy1.1-positive (with Thy1.1 screening tag on the knockout carrier) cells were aseptically separated by flow cytometry, which were the second-generation CAR19T_{IF} cells; the separated second-generation CAR19T_{IF} cells were again infused back into B6 mice, and the CAR19T_{IF} cells separated again were the third-generation CAR19T_{IF} cells.

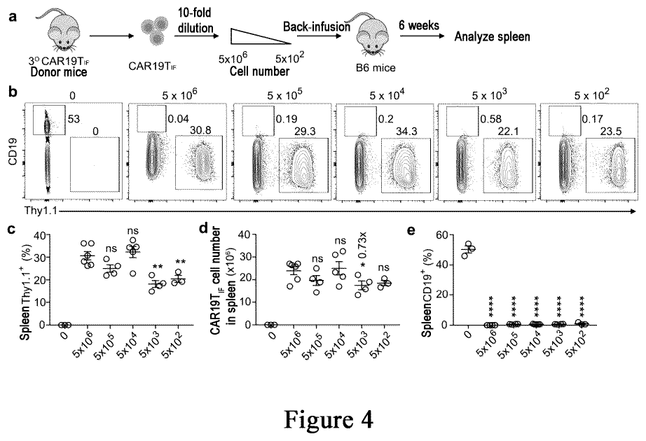

The experimental design procedure of infusing back the third-generation CAR19T_{IF} cells into B6 mice in a 10-fold gradient dilution was shown in FIG. 4a: the third-generation CAR19T_{IF} cells were diluted with PBS to obtain cell suspensions of different concentrations, so that the number of cells was 5×10⁶-5×10²; the cell suspensions of different concentrations were then infused back into B6 mice through the mice tail; 3-6 mice were back-infused for each concentration. Six weeks after the back-infusion, the proportion and number of CAR19T_{IF} cells (i.e., Thy1.1+) in the total spleen cells in the spleen of B6 recipient mice infused back with CAR19T_{IF} cells at various concentrations were analyzed by flow cytometry using the Thy1.1 antibody.

Six weeks after the back-infusion, the proportion and number of CD19+ cells and Thy1.1+ CAR19T_{IF} in the spleen of mice were analyzed and detected using flow cytometry. The results were shown in FIGS. 4b, 4c, and 4d. Despite the 10,000-fold difference in the cell input, CAR19T_{IF} cells showed similar percentages and cell numbers between different groups, indicating that CAR19T_{IF} cells have saturable properties in vivo. At the same time, as few as only 500 CAR19T_{IF} cells were sufficient to massively expand and eliminate all hundreds of millions of CD19+B cells in mice without any pretreatment, demonstrating the superior functions of CAR19T_{IF} cells.

The above results showed that CAR19T_{IF} cells had almost unlimited self-renewal ability like stem cells, and only a few cells were needed to massively expand and kill target cells.

### Example 5: Back-Infusion of CAR19T_{IF} Under the Condition of No Pretreatment Inhibited the Growth of MC38 Colon Cancer Tumors and Prolonged the Survival of Tumor-Bearing Mice

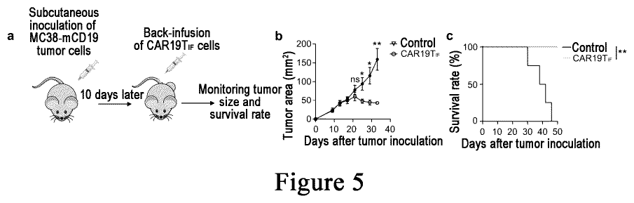

FIG. 5a was an experimental flow chart, the details were as follows:

An MC38 cell line expressing mCD19 was generated, namely MC38-mCD19.

Construction of pMSCV-mCD19-IRES-GFP recombinant plasmid: first of all, the carrier pMSCV (Addgene #162750) was digested using the restriction endonucleases XhoI (NEB #R0146L) and HpaI (NEB #R0105S), and the pMSCV plasmid backbone DNA was obtained by gel recovery; CD19 carrying the corresponding restriction sites of C57BL/6 mice (see UniProtKB-P25918 (CD19_MOUSE)) was obtained by PCR amplification taking the peripheral blood cDNA of C57BL/6 mice as a template and through the Q5 polymerase (NEB #M0491L) system; the purified CD19 cDNA coding sequence in C57BL/6 mice carrying the corresponding restriction sites and the pMSCV plasmid backbone DNA were ligated via the Blunt TA ligase (NEB #M0367L) to obtain the recombinant plasmid. Finally, upon the restriction digestion identification and sequencing confirmation, the pMSCV-mCD19-IRES-GFP recombinant plasmid was obtained.

According to the method in Example 1, the pMSCV-mCD19-IRES-GFP recombinant plasmid and pCL-Eco were co-transfected into Phoenix-Eco cells to prepare a retrovirus expressing mCD19 (pMSCV-mCD19-IRES-GFP); then the retrovirus expressing mCD19 (pMSCV-mCD19-IRES-GFP) was transfected into MC38 cells (ATCC #CRL-2599), and GFP and mCD19 double-positive cells were sorted and expanded, namely MC38-mCD19.

6-8 week-old B6 mice weighing 20-25 g were divided into two groups, namely the control group (4 mice) and the CAR19T_{IF} group (4 mice). Each mouse in each group was subcutaneously inoculated with 2×10⁵ MC38-mCD19 tumor cells. Ten days after inoculation: the CAR19T_{IF} cells obtained by infection with retrovirus prepared in Example 1 for 24 hours were prepared into a cell suspension with PBS, and infused back into each mouse in the CAR19T_{IF} group through the mice tail. 3×10⁶ CAR19T_{IF} cells were infused into each mouse through the tail vein of the mice; the same volume of PBS was infused into each mouse in the control group. Thereafter, the tumor size (tumor area mm²) of all mice and the final survival rate of mice were measured every three days (the experimental procedure was shown in FIG. 5a).

The tumor sizes of mice in each group were summarized according to different times after inoculation of tumor cells. The results were shown in FIG. 5b, compared with the tumor area of the mice in the control group, the tumor area of the mice infused back with CAR19T_{IF} cells was significantly reduced, indicating that the mCD19CAR cells with knockout of Bcor and Zc3h12a can significantly inhibit tumor growth.

The survival rates of mice in each group were summarized according to different times after inoculation of tumor cells. The results were shown in FIG. 5c. It can be seen that CAR19T_{IF} cells not only significantly inhibited tumor growth, but also greatly prolonged the survival period of the colon cancer model mice.

The above results showed that after the mice in the control group were subcutaneously inoculated with MC38-mCD19 colon tumor cells, MC38-mCD19 tumor cells rapidly formed tumors and grew subcutaneously. After the infusion of CAR19T_{IF}, the growth of the mice tumor was significantly inhibited, and the survival period of mice was greatly prolonged.

### Example 6. CAR19T_{IF} had an Immune Memory Protective Effect on Tumors, Preventing the Tumor Recurrence. Mice Pre-Inoculated with CAR19T_{IF} Inhibited the Growth of Colon Cancer MC38 and Melanoma B6F10-mCD19 Lung Metastasis Tumor Load and Prolonged the Survival Period of Tumor-Bearing Mice

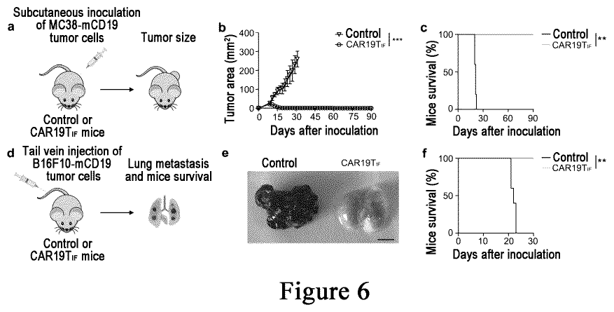

FIG. 6a was a flow chart:

### 1. Colon Cancer Model

In order to generate an MC38 cell line expressing mCD19, namely MC38-mCD19: transfect MC38 cells with the mCD19-expressing retrovirus pMSCV-mCD19-IRES-GFP, sort and expand GFP+ and mCD19 double-positive MC38 cells.

6-8 week-old B6 mice weighing 20-25 g were divided into 2 groups:
Control-MC38 group (5 mice): PBS was infused.
CAR19T_{IF}-MC38 group (10 mice): mCD19CAR cells (CAR19T_{IF}) with knockout of Bcor and Zc3h12a obtained by infection with the retrovirus prepared in Example 1 for 24 hours were formulated into a cell suspension with PBS, and infused back into each mouse in the CAR19T_{IF}-MC38 group through the mice tail, each mouse was infused back into 4×10⁵ CAR19T_{IF} cells through the tail;
One month later, each mouse in each group was subcutaneously inoculated with 2×10⁵ MC38-mCD19 tumor cells.

The tumor size (tumor area mm²) of all mice and the final survival rate of the mice were measured every three days in the colon cancer MC38 experiment.

The tumor sizes of mice in each group were summarized according to different times after inoculation of tumor cells. The results were shown in FIG. 6b, compared with the control group, the tumor area of the mice infused back with mCD19CAR cells (CAR19T_{IF}) with simultaneous knockout of Bcor and Zc3h12a was significantly reduced, indicating that the mCD19CAR cells with knockout of Bcor and Zc3h12a can significantly inhibit tumor growth.

The survival rates of mice in each group were summarized according to different times after inoculation of tumor cells. The results were shown in FIG. 6c. It can be seen that compared with the survival rate of the mice in the control group, back-infusing mCD19CAR cells (CAR19T_{IF}) with simultaneous knockout of Bcor and Zc3h12a not only significantly inhibited tumor growth, but also greatly prolonged the survival period of the colon cancer model mice.

### 2. Melanoma Model

The flow chart was shown in FIG. 6d.

In order to generate the B6F10 cell line expressing mCD19 (from ATCC, CRL-6475), namely B6F10-mCD19: B6F10 cells (ATCC Cat #CRL-6475) were transfected with the mCD19-expressing retrovirus pMSCV-mCD19-IRES-GFP, and GFP+ and mCD19 double-positive B6F10 cells were sorted and expanded.

6-8 week-old B6 mice weighing 20-25 g were divided into 2 groups:
Control-B6F10 group (5 mice): PBS was infused.
CAR19T_{IF}-B6F10 group (10 mice): CAR19T_{IF} cells obtained by infection with the retrovirus prepared in Example 1 for 24 hours were formulated into a cell suspension with PBS, and infused back into each mouse in the CAR19T_{IF}-B6F10 group through the mice tail, each mouse was infused back into 4×10⁵ CAR19T_{IF} cells through the tail;
One month later, 1×10⁵ B6F10-mCD19 tumor cells were injected into each mouse in each group through the mice tail.

Three weeks later, the lung tumor burden of the mice was checked, and the survival of the mice was monitored.

Three weeks later, the lung tissue of the mice was taken, and the results of detecting the lung tumor burden of the mice were shown in FIG. 6e. It can be seen that compared with the control group, no melanoma was enriched in lung tissue after back-infusion of mCD19CAR cells (CAR19T_{IF}) with simultaneous knockout of Bcor and Zc3h12a, indicating that CAR19T_{IF} inhibited melanoma lung metastasis.

The survival rates of mice at different times after inoculation of B6F10-mCD19 cells were summarized. The results were shown in FIG. 6f. It can be seen that compared with the control group, back-infusing mCD19CAR cells (CAR19T_{IF}) with simultaneous knockout of Bcor and Zc3h12a not only significantly inhibited the growth and metastasis of tumors, but also greatly prolonged the survival period of the melanoma model mice.

The above results showed that mice pre-injected with a small amount of CAR19T_{IF} cells can still effectively block the growth and metastasis of transplanted MC38 tumor cells or melanoma B16F10-mCD19 after a few weeks, indicating that CAR19T_{IF} cells have a memory effect and can provide long-term immune memory against tumors.

### Example 7. Construction and Identification of CAR19T_{IF} Cells Induced by Using CAR Targeting Human hCD19 Molecules

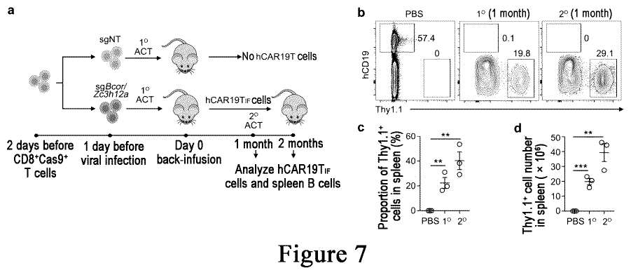

FIG. 7a was a schematic diagram of the experimental design of continuous transfer of hCAR19T_{IF} cells in humanized CD19 (hCD19) mice, the details were as follows:

CD8 T cells were isolated from the spleen and lymph nodes of Cas9+B6 mice, and after activated via CD3/CD28 for 24 hours, the activated CD8 T cells were obtained (the method was the same as that in section 2 in Example 1); then the retrovirus obtained by transfection with pMSCV-hU6-sgNT-EFS-Thy1.1-P2A-human CD19-CAR or pMSCV-hU6-sgBcor-mU6-sgZc3h12a-EFS-Thy1.1-P2A-human CD19-CAR was used to infect the activated CD8 T cells respectively, named as sgNT-hCD19CAR and hCAR19T_{IF} (sgZc3h12a/sgBcor) cells respectively, the above-mentioned cells obtained after infection for 24 hours were respectively infused into humanized CD19 transgenic B6 mice (hCD19) through the tail vein of mice. One month later, hCAR19T_{IF} were detected and isolated in the first-generation recipient mice and infused into new hCD19 mouse recipients (i.e., the second-generation recipients) through the mice tail. One month later, detection was performed by flow cytometry analysis.

The details were as follows:

### 1. Construction of Humanized Gene Knockout Carrier

This example constructed a retrovirus-based sgRNA expression vector, namely, pMSCV-hU6-sgNT-EFS-Thy1.1-P2A-humanized CD19-CAR and pMSCV-hU6-sgBcor-mU6-sgZc3h12a-EFS-Thy1.1-P2A-humanized CD19-CAR;
pMSCV-hU6-sgNT-EFS-Thy1.1-P2A-humanized CD19-CAR differed from pMSCV-hU6-sgNT-EFS-Thy1.1-P2A-CD19-CAR in Example 1 only in that the mouse CD19-CAR at position 1265-2692 of SEQ ID NO:1 was replaced with humanized CD19-CAR; the nucleotide sequence of the humanized CD19-CAR was SEQ ID No:8 (Hu19-CD828Z).
pMSCV-hU6-sgBcor-mU6-sgZc3h12a-EFS-Thy1.1-P2A-humanized CD19-CAR differed from pMSCV-hU6-sgBcor-hU6-sgZc3h12a-EFS-Thy1.1-P2A-CD19-CAR in Example 1 only in that the mouse CD19-CAR in the carrier was replaced with the humanized CD19-CAR; the nucleotide sequence of the humanized CD19-CAR was the cDNA sequence of hCD19-CAR (Hu19-CD828Z).

### 2. Isolation and Activation of Initial CD8 T Cells

Same as that in section 1 of Example 1.

3. Construction of Humanized CD19-CAR Cells with Knockout of Bcor and Zc3h12a

The same as that in section 1 of Example 1, the only difference was that the retroviral vector pMSCV-hU6-sgBcor-mU6-sgZc3h12a-EFS-Thy1.1-P2A-humanized CD19-CAR replaced pMSCV-hU6-sgBcor-hU6-sgZc3h12a-EFS-Thy1.1-P2A, and humanized CD19CAR cells with simultaneous knockout of Bcor and Zc3h12a (expressed as sgBcor/Zc3h12a) were obtained, named as hCAR19T_{IF}.

4. Construction of CD8 T Cells without Knocking Out Genes

The same as that in section 1 of Example 1, the only difference was that the retroviral vector pMSCV-hU6-sgNT-EFS-Thy1.1-P2A-humanized CD19-CAR replaced pMSCV-hU6-sgNT-EFS-Thy1.1-P2A-CD19-CAR to obtain humanized CD19CAR cells without knocking out genes (sgNT-hCD19CAR).

The above method of cell identification was the same as that in Example 1.

5. The Knockout of Zc3h12a and Bcor Genes Makes the Back-Infused hCAR19T_{IF} Cells Efficiently Expand Under the Condition of No Pretreatment

hCAR19T_{IF} obtained 24 hours after the infection of activated CD8T cells with the above retrovirus, was infused into humanized CD19 transgenic B6 mice (hCD19 mice) through the tail vein of mice. The details were as follows:
6-8 week-old hCD19 mice weighing 20-25 g were divided into 2 groups, namely sgNT group (3 mice) and sgBcor/Zc3h12a group (3 mice);
sgNT group: the above-mentioned hCD19CAR cells without knocking out genes (sgNT-hCD19CAR) were formulated into a cell suspension with PBS and infused back into each mouse in the sgNT group through the mice tail, and each mouse was infused back into 4×10⁵ sgNT-hCD19CAR cells without knocking out genes through the tail;
sgBcor/Zc3h12a group: the above-mentioned hCAR19T_{IF} cells were formulated into a cell suspension with PBS and infused back into each mouse in the sgBcor/Zc3h12a group through the mice tail, and each mouse was infused back into 4×10⁵ hCD19CAR cells with knockout of Bcor and Zc3h12a genes through the tail.

T cells were not infused back in the PBS group, an equal volume of PBS was infused as a treatment control.

One month later, hCAR19T_{IF} was detected and isolated in the first-generation recipient mice, and then infused into new hCD19 mouse recipients (i.e., the second-generation recipients) through the mice tail again in accordance with the first-generation infusion manner. One month later, flow cytometry analysis and detection was performed.

One month after the first-generation back-infusion and the second-generation back-infusion, the proportions of Thy1.1+ and hCD19+ positive B cells (APC anti-human CD19, Biolegend #302212) in the peripheral blood of mice were analyzed by flow cytometry, respectively. The results were shown in FIG. 7b. It can be seen that under the condition that no pretreatment was performed on the mice, there were no Thy1.1 positive cells in the PBS control group, and 57.4% of the spleen cells were hCD19+B cells; in the first generation (1° in the figure), about 19.8% of the cells in the spleen were Thy1.1+hCAR19T_{IF}, and 0.1% of them were hCD19+B cells; in the second generation (2° in the figure), about 29.1% of the cells in the spleen were Thy1.1+hCAR19T_{IF}, and 0% of them were hCD19+B cells.

One month after the first-generation back-infusion and the second-generation back-infusion, statistical analysis was performed on the proportion and the absolute number of hCAR19T_{IF} in the spleen of mice analyzed by flow cytometry, respectively. The results were shown in FIGS. 7c and 7d. It can be seen that under the condition that no pretreatment was performed on the mice, there were no Thy1.1 positive cells in the PBS control group, in the first generation (1° in the figure), about 20% of the cells in the spleen were Thy1.1 positive, in the second generation (2° in the figure), about 40% of the cells in the spleen were Thy1.1 positive cells (i.e., hCAR19T_{IF} cells).

The above results showed that, hCAR19-T_{IF} cells (i.e., hCAR19T_{IF}) can maintain the stem cell-like activity thereof in humanized CD19 mice, which was consistent with mCAR19-T_{IF} cells (i.e., CAR19T_{IF}), hCD19CART cells with simultaneous knockout of both Zc3h12a and Bcor genes exhibited almost unlimited self-renewal ability same as that of stem cells, but retained the functions of mature T cells.

### Example 8. Construction of mCD19CAR (CAR19T_{IF}-IL23R) Cells Expressing Secretory IL23R Fusion Proteins by CAR19T_{IF} and the Inhibitory Effect of Adoptive Cell Transfer Therapy of CAR19T_{IF}-IL23R on Dextran Sulfate-Induced Enteritis in Mice

### 1. Preparation of CAR19T_{IF}-IL23R Cells

1) Construction of pMSCV-EFS-spIl2-IL23R-mIgG2a-Fc Recombinant Plasmid

First of all, a secretory IL23R-mlgG2a-Fc plasmid was obtained by the method of gene synthesis. The sequence of IL2 secretory peptide-IL23R-mIgG2a-Fc fusion protein was SEQ ID NO: 9. The recombinant plasmid was obtained. Finally, upon the enzyme digestion identification and sequencing confirmation, the pMSCV-EFS-spIl2-IL23R-mIgG2aFc recombinant plasmid was obtained.

### 2) Activation of CD8 T Cells

Same as that in section 2 of Example 1: CD8 T cells were isolated from the spleen and lymph nodes of Cas9+B6 mice, activated via CD3/CD28 for 24 hours, and activated CD8 T cells were obtained;

### 3) CAR19T_{IF}-IL23R Cells

Phoenix-Eco cells were transfected with pMSCV-hU6-sgBcor-hU6-sgZc3h12a-EFS-Thy1.1-P2A-CD19-CAR to obtain a retrovirus expressing pMSCV-hU6-sgBcor-hU6-sgZc3h12a-EFS-Thy 1.1-P2A-CD19-CAR;
Phoenix-Eco cells were transfected with pMSCV-EFS-spIl2-IL23R-mIgG2aFc to obtain a retrovirus expressing pMSCV-EFS-spIl2-IL23R-mIgG2aFc;
The above two retroviruses were then co-infected the activated CD8 T cells to obtain recombinant cells named as CAR19T_{IF}-IL23R. 24 hours after the infection, the infected cells were infused into B6 mice via the tail vein of mice.

The above CAR19T_{IF}-IL23R can also be obtained by transfecting CAR19T_{IF} cells with pMSCV-EFS-spIl2-IL23R-mIgG2aFc through retrovirus.

### 2. Western Blot Protein Detection

The cell culture supernatant and cells of the CAR19T_{IF}-IL23R cells prepared in the above section 1 were harvested respectively, the cells were lysed on ice with the cell lysis buffer RIPA. Subsequently, after centrifugation at 4° C. and 12,000 rpm, the supernatant of the cell lysate was carefully aspirated. The protein sample was separated by SDS-PAGE and then transferred to a PVDF membrane using a semi-dry transfer apparatus. Subsequently, after the PVDF was blocked, the antibody was incubated and then the color was developed. The CAR19T_{IF} cells prepared in Example 1 were taken as a control.

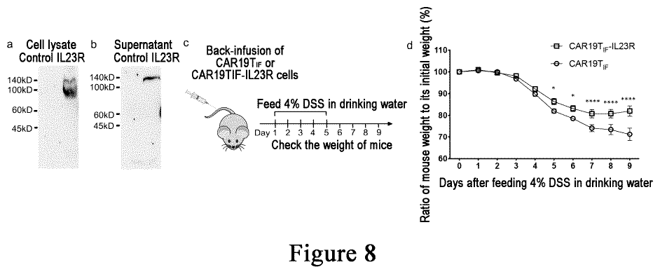

The results were shown in FIGS. 8a and 8b. It can be seen that compared with the control, the CAR19T_{IF}-IL23R cells (represented by IL23R in the figure) secreted the IL23R protein.

### 3. The Back-Infusion of CAR19T_{IF}-IL23R Repressed Dextran Sulfate-Induced Enteritis

The procedure was shown in FIG. 8c, the details were as follows:
8-10 week-old B6 mice weighing about 25 g were divided into 2 groups:
CAR19T_{IF} group (5 mice): 1×10⁶ CAR19T_{IF} cells were infused back into B6 mice through the tail vein of mice, the specific method was the same as before.
CAR19T_{IF}-IL23R group (5 mice): 1×10⁶ CAR19T_{IF}-IL23R cells were infused back into B6 mice through the tail vein of mice, the specific method was the same as before.

Four weeks after the cell back-infusion, the mice were fed with 4% dextran sulfate (DSS) for 5 days to induce and initiate enteritis.

After feeding, the mice were weighed daily, and the ratios of the weights of the mice at different times to their initial weights were calculated; the physical signs of the mice were observed and whether the feces of the mice were abnormal or not was monitored.

The results of the ratios of the weights of the mice at different times after feeding to their initial weights were shown in FIG. 8d. It can be seen that compared with the group in which CAR19T_{IF} were back-infused, the weight of the mice infused back with CAR19T_{IF}-IL23R cells decreased, indicating that IL23R-secreting CAR19T_{IF}-IL23R cells can significantly inhibit dextran sulfate-induced enteritis.

### Example 9. Simultaneous Knockout of Bcor and Zc3h12a Promoted the Expansion and Persistence of GD2 CAR-T Cells (GD2T_{IF}). Knockout of Bcor or Zc3h12a Alone Cannot Promote the Expansion of GD2 CAR-T Cells

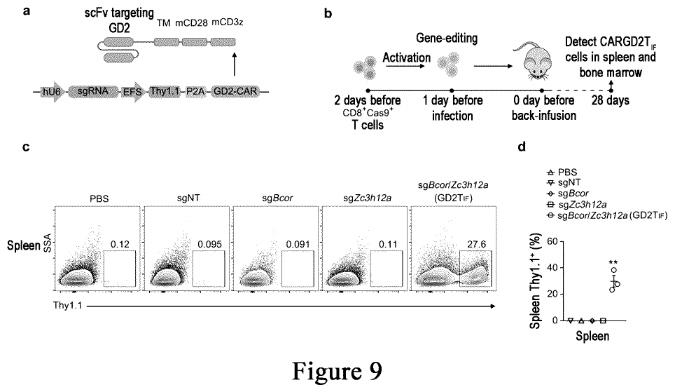

FIG. 9a showed the structure of GD2 CAR, wherein the scFv targeting the GD2 antigen was a scFv protein that recognized GD2 and derived from the monoclonal antibody 14 g2, the nucleic acid sequence thereof was SEQ ID NO:10 (see “A chimeric T cell antigen receptor that augments cytokine release and supports clonal expansion of primary human T cells.” Mol. Ther. 12, 933-941 (2005)).

The flow chart was shown in FIG. 9b. The preparation method of GD2 CAR-T cells was the same as that of Example 1, only the scFv used was different.

28 days after the cells were infused, the proportion of GD2 CAR-T cells in the spleen was detected. The results were shown in FIGS. 9c and 9d. Only GD2 CAR-T with simultaneous knockout of Bcor and Zc3h12a can expand in mice under the condition of no pretreatment, knockout of Bcor or Zc3h12a alone had no effect. GD2 CAR-T cells with simultaneous knockout of Bcor and Zc3h12a were named as GD2T_{IF}.

### Example 10. GD2T_{IF} had Properties of Stem Cells and can be Passaged in B6 Mice and NSG Mice while Retaining T Cell Functions; However, it would not Form Tumors in Mice and Possessed Safety

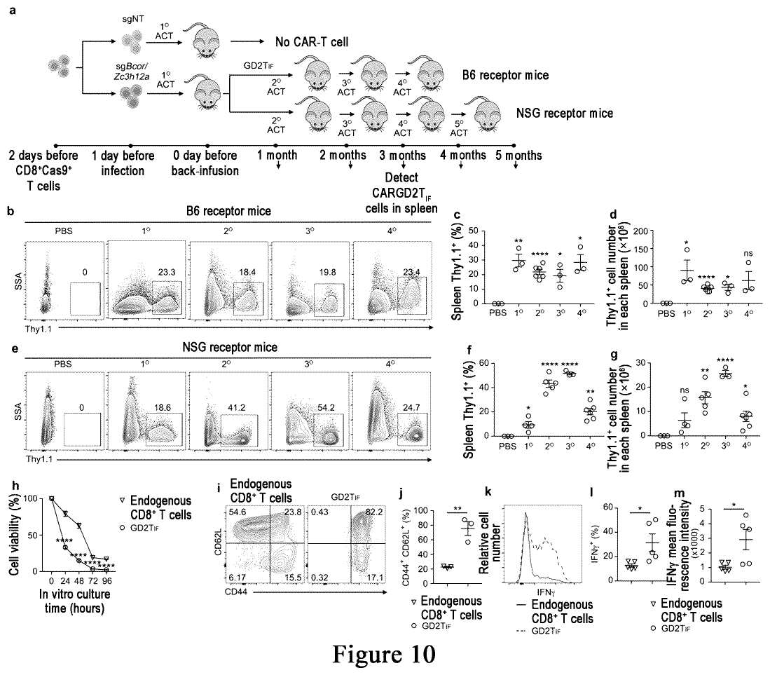

The flow chart was shown in FIG. 10a. The preparation method of GD2 CAR-T cells was the same as that in Example 1. The first-generation GD2T_{IF} was infused into the second-generation mice under the condition of no pretreatment, and the experiment was repeated for the third and fourth generations of mice. The first-generation recipient mice were B6 mice. From the second generation, the recipient mice were divided into two types, one type was B6 mice and the other one was immunodeficient NSG (purchased from Shanghai Model Organisms).

One month after the infusion of cells of each generation, the proportion and number of GD2T_{IF} in the spleen (FIGS. 10b-10g), as well as the phenotypes (FIGS. 10i and 10j) and functions (FIGS. 10k-10m) were detected. The results were shown in FIGS. 10b-10g. Under the condition of no pretreatment, GD2T_{IF} can be repeatedly passaged in B6 mice and NSG mice, indicating that GD2T_{IF} had real stemness, which was similar to CAR19T_{IF} (Example 3). The results of this experiment showed that the T cell stemness induced by the simultaneous knockout of Bcor and Zc3h12a was universal and not limited to a specific CAR. At the same time, GD2T_{IF} cannot form tumors in highly immunodeficient NSG mice, indicating that GD2T_{IF} did not transform into tumor cells and possessed safety.

As shown in FIG. 10h, GD2T_{IF} cannot survive in vitro, indicating that GD2T_{IF} did not transform into tumor cells and possessed safety.

As shown in FIGS. 10i and 10j, flow cytometry analysis showed that GD2T_{IF} exhibited a CD44+CD62L+ memory T cell phenotype, which was consistent with its stem cell nature.

As shown in FIGS. 10k-10m, flow cytometry analysis showed that GD2T_{IF} was able to secrete IFNg, indicating that GD2T_{IF} had T cell functions.

### Example 11. Simultaneous Knockout of Bcor and Zc3h12a Promoted the Expansion and Persistence of EGFR CAR-T Cells (EGFRT_{IF}), Knockout of Bcor or Zc3h12a Alone Cannot Promote the Expansion of EGFR CAR-T Cells

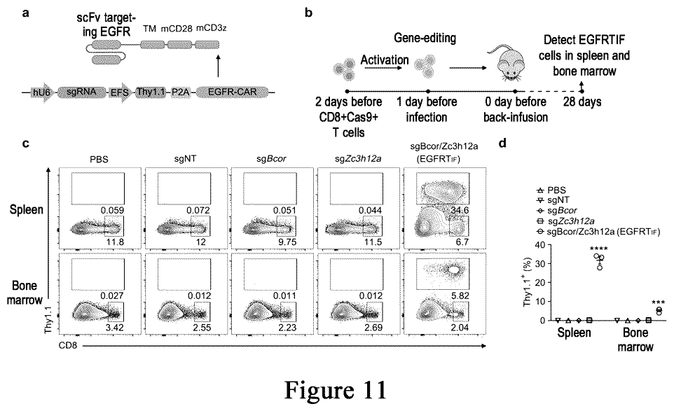

FIG. 11a showed the structure of EGFR CAR, wherein the scFv targeting EGFR antigen was a scFv protein that recognized EGFR and derived from the monoclonal antibody Cetuximab, and the nucleotide sequence thereof was SEQ ID NO:11 (see H. G. Caruso, L. V. Hurton, A. Najjar, D. Rushworth, S. Ang, S. Olivares, T. Mi, K. Switzer, H. Singh, H. Huls, D. A. Lee, A. B. Heimberger, R. E. Champlin, L. J. N. Cooper, Tuning sensitivity of CAR to EGFR density limits recognition of normal tissue while maintaining potent antitumor activity. Cancer Res. 75, 3505-3518 (2015)). The experimental flow chart was shown in FIG. 11b, the preparation method of EGFR CAR-T cells was similar to that of Example 1. 28 days after the infusion of cells, the proportion of EGFR CAR-T cells in the spleen and bone marrow was detected. The results were shown in FIGS. 11c and 11d. Only EGFR CAR-T with simultaneous knockout of Bcor and Zc3h12a can expand in mice under the condition of no pretreatment, knockout of Bcor or Zc3h12a alone had no effect. EGFR CAR-T cells with simultaneous knockout of Bcor and Zc3h12a were named as EGFRT_{IF}.

### Example 12. EGFRT_{IF} Cells had the Properties of Stem Cells, and can be Passaged in B6 Mice while Retaining T Cell Functions; However, they would not Form Tumors in Mice and Possessed Safety

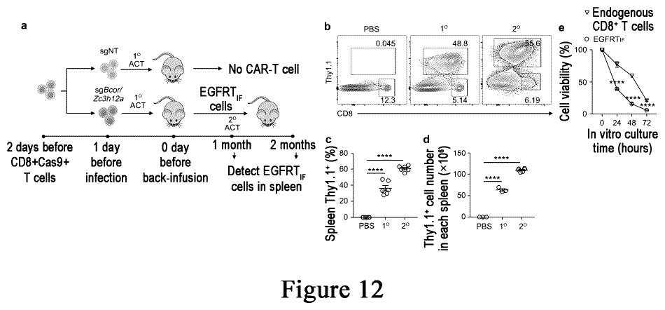

The flow chart was shown in FIG. 12a. The preparation method of EGFR CAR-T cells was the same as that in Example 9. The first-generation GD2T_{IF} were infused into the second-generation mice under the condition of no pretreatment.

One month after the infusion of cells of each generation, the proportion and number of GD2T_{IF} in the spleen were detected. As shown in FIGS. 12b-12d, EGFRT_{IF} can be passaged in B6 under the condition of no pretreatment, indicating that EGFRT_{IF} had stemness, which was similar to CAR19T_{IF} (Example 3). This experimental result once again showed that the T cell stemness induced by the simultaneous knockout of Bcor and Zc3h12a was universal and not limited to a specific CAR.

As shown in FIG. 12e, GD2T_{IF} cannot survive in vitro, indicating that GD2T_{IF} did not transform into tumor cells and possessed safety.

### Example 13. EGFRT_{IF} Inhibited Tumor Growth in Tumor-Bearing Mice Under the Condition of No Pretreatment

As shown in Example 11, both wild-type EGFR CAR-T cells and EGFR CAR-T cells with knockout of Bcor or Zc3h12a alone could not expand in vivo in mice with normal immunity under the condition of no pretreatment (FIG. 11). Therefore, in the present example, these three types of cells were not used as controls, only PBS was taken as a control.

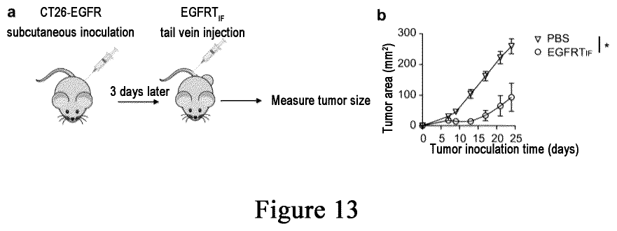

FIG. 13a was an experimental flow chart, the details were as follows:

Preparation of CT26 cell line expressing EGFR, i.e., CT26-EGFR. Construction of LentiCas9-EGFR-T2A-Thy1.1 recombinant plasmid: first of all, the carrier lentiCas9-Blast (Addgene #52962) was digested with restriction endonucleases AgeI (NEB #R3552L) and EcoRI (NEB #R3101L), and the LentiCas9 plasmid backbone DNA was obtained by gel recovery; EGFR-T2A-Thy1.1 cDNA coding sequence carrying the corresponding restriction site was obtained by nested PCR amplification taking the plasmids pHAGE-CMV-EGFR-puro and pMIG-hU6-sgNT-EFS-Thy1.1-mouse CD19-CAR as templates through the Q5 polymerase (NEB #M0491L) system; the purified EGFR-T2A-Thy1.1 cDNA coding sequence carrying the corresponding restriction site and the LentiCas9 plasmid backbone DNA were ligated via the Blunt TA ligase (NEB #M0367L) to obtain a recombinant plasmid. Finally, upon the restriction digestion identification and sequencing confirmation, the LentiCas9-EGFR-T2A-Thy1.1 recombinant plasmid was obtained.

The LentiCas9-EGFR-T2A-Thy1.1 recombinant plasmid and the viral packaging plasmid psPAX2/pMD2.G were co-transfected into 293T cells to prepare EGFR-expressing lentivirus (LentiCas9-EGFR-T2A-Thy1.1); then the EGFR-expressing retrovirus (LentiCas9-EGFR-T2A-Thy1.1) was transfected into CT26 cells (ATCC #CRL-2638), and Thy1.1-positive cells were sorted and expanded, namely CT26-EGFR.

The 6-8 week-old Balb/c and B6 F1 mice weighing 20-25 g were divided into two groups, namely the control group (8 mice) and the EGFRT_{IF} group (6 mice), and each mouse in each group was subcutaneously inoculated with 1×10⁶ CT26-EGFR tumor cells. Three days after tumor inoculation, EGFRT_{IF} cells were prepared in a similar method to that of Example 11, wherein CD8 T cells were from the F1 generation of Balb/c and B6 mice. EGFRT_{IF} was formulated into a cell suspension with PBS and infused back into each mouse in the EGFRT_{IF} group through the mice tail, each mouse were infused with 7×10⁵ CAR19T_{IF} cells through the tail vein of mice; the same volume of PBS was infused into each mouse in the control group. Thereafter, the tumor sizes (tumor area mm²) of all mice were measured every three days (the experimental procedure was shown in FIG. 13a).

The tumor sizes of mice in each group were summarized according to different times after inoculation of tumor cells. The results were shown in FIG. 13b, compared with the tumor area of the mice in the control group, the tumor area of the mice infused back with EGFRT_{IF} cells was significantly reduced. EGFRT_{IF} cells can significantly inhibit tumor growth.

The above results showed that after the mice in the control group were subcutaneously inoculated with CT26-EGFR colon tumor cells, CT26-EGFR tumor cells quickly formed tumors and grew subcutaneously. After the EGFRT_{IF} cells were infused, the growth of the mice tumor was significantly inhibited.

### Example 14. GD2T_{IF} as a Carrier to Continuously Secrete TNF In Vivo to Induce a Disease Model of Chronic Inflammation (GD2T_{IF}-TNF)

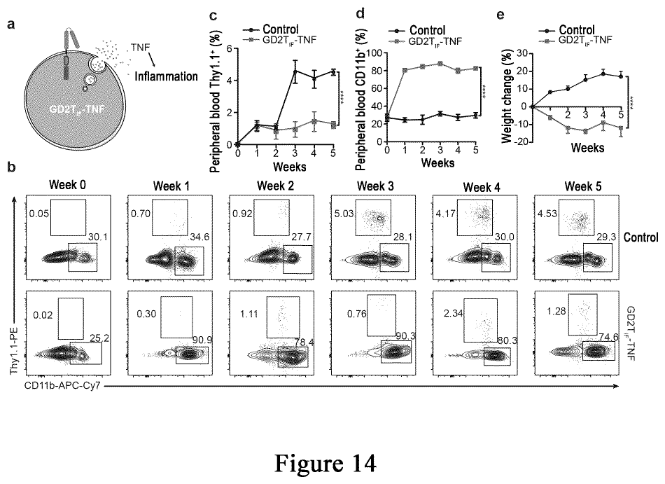

FIG. 14a was a schematic diagram of the experimental principle, which was a method for simply and quickly establishing an inflammatory disease model by overexpressing the inflammatory factor TNF in GD2T_{IF} cells. The preparation method of GD2T_{IF} was similar to that of Example 9, it was infected with a virus that overexpresses TNF meanwhile in the preparation process. The present invention constructed the pMSCV-EF1a-GFP-P2A-TNF recombinant plasmid. Wherein, the human TNF was referred to UniProtKB-P01375 (TNFA_HUMAN). The virus with knockout of genes and the pMSCV-EF1a-GFP-P2A-TNF virus were co-infected the mouse T cells, and Thy1.1+GFP+ double-positive CD8 T cells were GD2T_{IF}-TNF. GD2T_{IF}-TNF or GD2T_{IF} (control) were infused back into mice, the proportion of peripheral blood cells was detected, and the change in weights of mice was recorded.

The experimental results were shown in FIGS. 14b-14d. The mice infused with GD2T_{IF}-TNF showed a significant increase in inflammatory myeloid cells (CD11b+), and these mice lost weight (FIG. 14e). These results indicated that the back-infusion of GD2T_{IF}-TNF leads to chronic inflammation in mice. Therefore, GD2T_{IF} can be used as a cell carrier to continuously secrete inflammatory factors in vivo and be used to establish various disease models. The advantage of this method was that the cells needed to be infused only once, and repeated administration was not required.

### Example 15. GD2T_{IF} Cells as a Carrier to Continuously Secrete IL-5 to Induce Eosinophilia Model (GD2T_{IF}-IL-5)

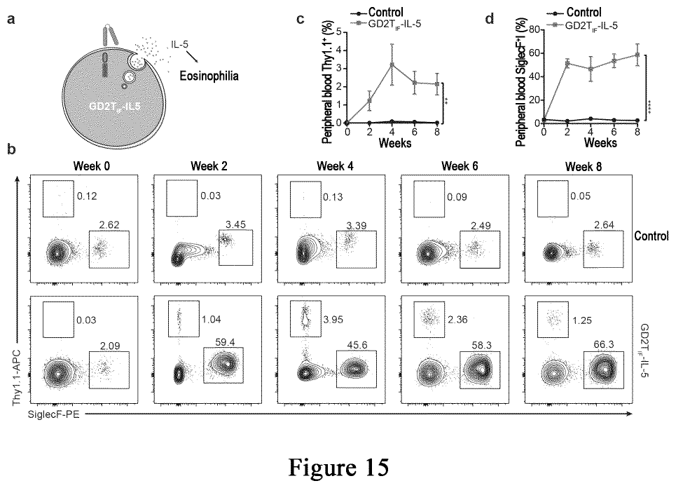

FIG. 15a was a schematic diagram of the experimental principle, which was a method for simply and quickly establishing an eosinophilia model by overexpressing the growth factor IL-5 of eosinophils in GD2T_{IF} cells. The overall implementation process was similar to that of Example 14. The preparation method of GD2T_{IF} was similar to that of Example 9, it was infected with a virus that overexpresses IL-5 meanwhile in the preparation process. The present invention constructed the pMSCV-EF1a-GFP-P2A-IL-5 recombinant plasmid. Wherein, the mouse IL-5 was referred to UniProtKB-P04401 (IL5_MOUSE). The virus with knockout of genes and the pMSCV-EF1a-GFP-P2A-IL-5 virus were co-infected the mouse T cells, and Thy1.1+GFP+ double-positive CD8 T cells were GD2T_{IF}-IL-5. GD2T_{IF}-IL-5 or GD2T_{IF} (control) were infused back into mice, and the proportion of peripheral blood cells was detected.

The experimental results were shown in FIGS. 15b-15d, the eosinophils (SiglecF+) in the peripheral blood of mice infused with GD2T_{IF}-IL-5 were significantly increased. Therefore, GD2T_{IF} can be used as a cell carrier to secrete various growth factors in vivo, which can be used to establish various disease models and treat diseases. The advantage of this method was that the cells needed to be infused only once, and repeated administration was not required.

### Example 16. GD2T_{IF} Cells as a Carrier to Continuously Secrete GLP1 to Treat Obesity and Diabetes (GD2T_{IF}-GLP1)

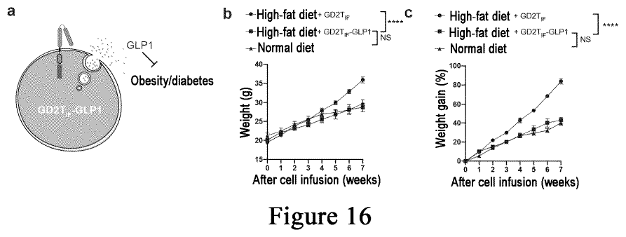

FIG. 16a was a schematic diagram of the experimental principle, which is used to treat obesity and diabetes by overexpressing GLP1 in GD2T_{IF} cells. GLP1 and the agonists of the receptor thereof had been approved by FDA for the treatment of obesity and diabetes, but all of these drugs require repeated administration. This example uses GD2T_{IF} cells to continuously secrete GLP1 in vivo to achieve the purpose of cure through a single administration.

The overall implementation process was similar to that of Example 14. The preparation method of GD2T_{IF} was similar to that of Example 9, it was infected with a virus that overexpresses GLP1 meanwhile in the preparation process. The present invention constructed the pMSCV-EF1a-GFP-P2A-GLP1 recombinant plasmid. The secretory GLP1 (sGLP1) sequence was whole gene synthesized, and the nucleotide sequence thereof was SEQ ID NO: 12, this sequence comprised a sequence with a point mutation in the DPP4 recognition site and was fused to the mIgG2a-Fc segment so as to increase the half-life of GLP1. The virus with knockout of genes and the pMSCV-EF1a-GFP-P2A-GLP1 virus were co-infected the mouse T cells, and Thy1.1+GFP+ double-positive CD8 T cells were GD2T_{IF}-GLP1. GD2T_{IF}-GLP1 or GD2T_{IF} (control) were infused back into 5-week-old mice, and the mice began to be fed a high-fat diet one week later. Mice of the same age fed a normal diet were used as the baseline for detecting the therapeutic effect.

The experimental results were shown in FIGS. 16b and 16c. After being fed a high-fat diet, the weight gain of mice infused with GD2T_{IF}-GLP1 was significantly lower than that of mice infused with GD2T_{IF}, and the weight gain of mice infused with GD2T_{IF}-GLP1 was no different from that of mice of the same age fed a normal diet. This result showed that GD2T_{IF}-GLP1 had a significant therapeutic effect on obesity caused by a high-fat diet. Only a single administration was required in this therapy, and the efficacy was long-lasting. Therefore, GD2T_{IF} can be used as a cell carrier to secrete various molecules with therapeutic effects in vivo for the treatment of various chronic diseases that required repeated administration. The advantage of this method was that the cells needed to be infused only once for long-term effectiveness, and repeated administration was not required.

All references, articles, publications, patents, patent disclosure and patent applications cited herein are incorporated herein by reference in their entirety for all purposes. However, the mention of any references, articles, publications, patents, patent disclosure and patent applications cited herein is not and should not be considered as an admission or any form of suggestion that they constitute available prior art or constitute part of the common knowledge in any country in the world.

1. A recombinant immune cell, wherein the expression and/or functions of the BCOR gene and the ZC3H12A gene are reduced or eliminated.

2. The recombinant immune cell according to claim 1, characterized in that: the immune cell is selected from one or more of T cells, B cells, NK cells, mast cells, and tumor-infiltrating lymphocytes, preferably T cells or NK cells; and wherein the T cell is selected from one or more of CD4+CD8+ T cells, CD8+T cells, CD4+T cells, effector T cells, suppressor T cells, primitive T cells, memory T cells, γ-δT cells, α-βT cells, CD4-CD8-double negative T cells or NKT cells.

3. (canceled)

4. The recombinant immune cell according to claim 1, wherein the BCOR gene and the ZC3H12A gene in the recombinant immune cell are treated with gene knockout technology, gene silencing technology, inactivation mutation technology, PROTAC technology or small molecule inhibitors; optionally wherein the expression or functions of the BCOR gene and/or the ZC3H12A gene are reduced by at least 50%, at least 60%, at least 70%, at least 80%, at least 90%, at least 95% or 100%, respectively, compared with unmodified or control immune cells.

5. (canceled)

6. The recombinant immune cell according to claim 1, wherein the recombinant immune cell further comprises one or more structures for adoptive cell transfer therapy, optionally wherein the structure for adoptive cell transfer therapy is a chimeric antigen receptor (CAR) structure, a T cell antigen receptor (TCR) structure, a receptor structure based on ligand-receptor binding or a synthetic T cell receptor and antigen receptor (STAR), optionally wherein the antigen bound by the antigen receptor is one or more of ROR1, Her2, L1-CAM, CD4, CD5, CD8, CD19, CD20, BCMA, CD7, Clauding 18.2, GPC3, MSLN, AFP, CD22, mesothelin, CEA, hepatitis B surface antigen, antifolate receptor, CD23, CD24, CD30, CD33, CD38, CD44, EGFR, EGFRVIII, EGP-2, EGP-4, EPHa2, ErbB2, ErbB3, ErbB4, FBP, fetal acetylcholine receptor, GD2, GD3, HMWMAA, IL-22R-α, IL-13R-α2, kdr, κ light chain, Lewis Y, L1-cell adhesion molecule (CD171), MAGE-A1, mesothelin, MUC1, MUC16, PSCA, NKG2D ligand, NY-ESO-1, MART-1, gp100, tumor embryonic antigen, TAG72, VEGF-R2, carcinoembryonic antigen (CEA), prostate specific antigen, PSMA, estrogen receptor, progesterone receptor, ephrin B2, CD123, CS-1, c-Met, MAGE A3, CE7, Wilms tumor 1 (WT-1), cyclin A1 (CCNA1), interleukin 12, or other tumor-associated antigens.

7-9. (canceled)

10. The recombinant immune cell according of claim 1, wherein the recombinant immune cell further comprises a gene expressing biological molecules for treating diseases, optionally wherein the biological molecule expressed for treating diseases is selected from the group consisting of cytokines, hormones, growth factors, coagulation factors, chemokines, co-stimulatory molecules, activation peptides, antibodies or antigen-binding fragments thereof; optionally wherein the biological molecule for treating diseases is selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, and GLP1.

11-12. (canceled)

13. The recombinant immune cell of claim 1, characterized in that: at least 1 week, 2 weeks, 3 weeks, 4 weeks, 1 month, 2 months, 3 months, 4 months, 5 months, 6 months, 9 months, 12 months, 18 months, 2 years, 5 years, 10 years, 20 years, or 40 years after administration to the subject, the recombinant immune cell can be detected in the peripheral blood of the subject and/or the proportion of the recombinant immune cells in which expression and/or functions of the BCOR gene and ZC3H12A gene are reduced or eliminated is not less than 20%, 30%, 40%, 50%, 60%, 70%, 80%, 90% or 95% relative to the total amount of immune cells of the same type; and/or the proportion of the recombinant immune cells in which expression and/or functions of the BCOR gene and ZC3H12A gene are reduced or eliminated is 1%-35%, 3-30% or 3-20% relative to the total number of peripheral blood cells.

14. (canceled)

15. A method for preparing the recombinant immune cell of claim 1, comprising treating the BCOR gene and ZC3H12A gene in the recombinant immune cell with gene silencing technology, inactivation mutation technology, small molecule inhibitors, or gene knockout technology; optionally wherein the gene knockout technology comprises CRISPR/Cas technology, artificial zinc finger nucleases (ZFN) technology, transcription activator-like effector (TALE) technology or TALE-CRISPR/Cas technology; optionally wherein the CRISPR/Cas technology is selected from the group consisting of CRISPR-Cas9, CRISPR-Cas3, CRISPR-CasX, CRISPR-IscB, CRISPR-Cas12a, CRISPR-Cas12b, CRISPR-Cas13a, CRISPR-Cas13b, CRISPR-Cas13c, CRISPR-Cas13e or CRISPR-Cas13f system.

16-18. (canceled)

19. The method for preparing recombinant immune cells according to claim 15, characterized in that: the CRISPR/Cas technology uses a Cas endonuclease and a guide RNA (gRNA) targeting the BCOR gene, and/or a Cas endonuclease and a gRNA targeting the ZC3H12A gene; optionally wherein the gRNA protospacer targeting the BCOR gene binds to a target DNA sequence having at least 90%, 95%, 96%, 97%, 98%, 99% or 100% identity with the DNA sequence encoded by the BCOR gene of the subject (NCBI Gene ID: 54880 or NCBI Gene ID: 71458); and
the gRNA protospacer targeting the ZC3H12A gene binds to a target DNA sequence having at least 90%, 95%, 96%, 97%, 98%, 99% or 100% identity with the DNA sequence encoded by the ZC3H12A gene of the subject (NCBI Gene ID: 80149 or NCBI Gene ID: 230738).

20-21. (canceled)

22. The method for preparing recombinant immune cells according to claim 19, wherein the gRNA protospacer targeting the BCOR gene comprises a sequence having at least 85%, 90%, 95%, or 100% identity with the sequence ACTGGGCAATACCGCAACAG (SEQ ID NO: 3); wherein the guide gRNA protospacer targeting the ZC3H12A gene comprises a sequence having at least 85%, 90%, 95%, or 100% identity with the sequence CTAGGGGAATTGGTGAAGCA (SEQ ID NO: 4).

23. The method for preparing recombinant immune cells according to claim 15, characterized in that: the sequence of a CAR structure, TCR structure, ligand-receptor structure, STAR structure or other corresponding structures of targeted adoptive cell transfer therapy; and/or biological molecules expressed for treating diseases are further introduced into the immune cell; optionally wherein, the biological molecules for treating diseases are selected from one or more of IL-23R protein, IL-4R antibody, IFN-α, IFN-β, IFN-γ, IL-2, IL-3, IL-4, IL-5, IL-6, IL-8, IL-12, IL-13, IL-22, IL-23, IL-24, TNF, TNF-α, GM-CSF, CD40L, CTLA-4, FLT3L, TRAIL, LIGHT, or GLP1.

24-25. (canceled)

26. The method for preparing recombinant immune cells according to claim 19, wherein a single sgRNA (sgRNA) expression vector comprises:
any one of the following vector-promoters expressing the sgRNA and a biological molecule for treating diseases comprising: 1-sgZc3h12a-promoter 2-tag-P2A-the biological molecule sequence for treating diseases, 1-sgBcor-promoter 2-tag-P2A-the biological molecule sequence for treating diseases or pMSCV-promoter 1-sgBcor-promoter 2-sgZc3h12a-promoter 3-tag-P2A-the biological molecule sequence for treating diseases;
optionally wherein the biological molecule sequence for treating diseases is the structure sequence of the adoptive cell transfer therapy, or the sequence corresponding to the biological molecules for treating diseases; wherein the sgRNA expression vector comprises the fundamental structure of pMSCV-hU6-sgZc3h12a-EFS-Thy1.1-P2A-CD19-CAR, pMSCV-hU6-sgBcor-EFS-Thy1.1-P2A-CD19-CAR or pMSCV-hU6-sgBcor-hU6-sgZc3h12a-EFS-Thy1.1-P2A-CD19-CAR.

27. The method for preparing recombinant immune cells according to claim 15, characterized in that: the expression vector is introduced into the recombinant immune cells; wherein the introduction comprises virus or phage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and/or microfluidics delivery method.

28-36. (canceled)

37. A kit comprising a gene regulation system for preparing the recombinant immune cells of claim 1.

38. A method for producing a recombinant immune cell, wherein the expression and/or functions of the BCOR gene and the ZC3H12A gene are reduced or eliminated, comprising:
(1) obtaining autologous or allogenic immune cells;
(2) treating the immune cells using the preparation method of claim 15;
(3) reducing or eliminating the expression and/or functions of the BCOR gene and the ZC3H12A gene in the immune cells; optionally wherein the immune cells are implanted into a subject for in vivo expansion, and following in vivo expansion, the immune cells are isolated from the subject.

39-41. (canceled)

42. A method for treating a disease or condition in a subject in need thereof, the method comprising administering to the subject the recombinant immune cells of claim 1, optionally wherein the disease or condition is cancer, autoimmune disease, infectious disease, inflammatory disease, metabolic disease, neurodegenerative disease, disease caused by exogenous CAR structure targeting cells, or a disease caused by exogenous TCR structure targeting cells; optionally wherein the disease or condition comprises one or more of the following: leukemia, lymphoma, chronic lymphocytic leukemia (CLL), acute lymphocytic leukemia (ALL), non-Hodgkin's lymphoma, acute myeloid leukemia, multiple myeloma, refractory follicular lymphoma, mantle cell lymphoma, indolent B-cell lymphoma, B-cell malignancies, colon cancer, lung cancer, liver cancer, breast cancer, prostate cancer, ovarian cancer, skin cancer, melanoma, bone cancer, and brain cancer, ovarian cancer, epithelial cancer, renal cell carcinoma, pancreatic cancer, Hodgkin's lymphoma, cervical cancer, colorectal cancer, glioblastoma, neuroblastoma, Ewing's sarcoma, medulloblastoma, osteosarcoma, synovial sarcoma, mesothelioma, ankylosing spondylitis (AS), psoriasis (PS), celiac disease (CEL), systemic lupus erythematosus (SLE), common variable immunodeficiency (CVID), inflammatory bowel disease (IBD), ulcerative colitis (UC), type I diabetes (TID), juvenile idiopathic arthritis (JIA), Crohn's disease (CD), alopecia areata (AA), multiple sclerosis (MS), primary biliary cirrhosis (PBC), primary sclerosing cholangitis (PSC), rheumatoid arthritis (RA), Sjogren's syndrome (SJO), systemic sclerosis (SSC), spondyloarthropathies (SPA), vitiligo (VIT), asthma, or thyroiditis (AITD, THY or TH).

43-46. (canceled)

47. A method of reducing or eliminating the expression and/or functions of BCOR gene and ZC3H12A gene in immune cells, wherein the method includes increasing the stemness of immune cells, inhibiting the exhaustion of immune cells, promoting the expansion of immune cells, conferring memory to immune cells, prolonging the persistence of immune cells, and increasing the self-renewal ability of immune cells; wherein the recombinant immune cell with reduced or eliminated expression/and or functions of the BCOR and ZC3H12A genes is the recombinant immune cell of claim 1.

48. (canceled)

49. A method for producing an animal model, characterized in that the immune cells of an animal are treated using the preparation method of claim 15.

50. An animal model produced using the method of claim 49.

51. (canceled)
